(19) United States
(12) Patent Application Publication
(10) Pub. No.: US 2013/0172308 A1
(43) Pub. Date: Jul. 4, 2013

(54) ANTIMICROBIAL AGENTS

(71) Applicants: Yanyan MULYANA, Rosslea (AU); Marshall Leonard Feterl, Belgian Gardens (AU); Frank Richard Keene, Condon (AU); Fangfei Li, Canberra (AU); John Grant Collins, Canberra (AU); Jeffrey Mitchell Warner, Toomulla (AU); Kirsten Ruth Maria Heimann, Condon (AU)

(72) Inventors: Yanyan MULYANA, Rosslea (AU); Marshall Leonard Feterl, Belgian Gardens (AU); Frank Richard Keene, Condon (AU); Fangfei Li, Canberra (AU); John Grant Collins, Canberra (AU); Jeffrey Mitchell Warner, Toomulla (AU); Kirsten Ruth Maria Heimann, Condon (AU)

(73) Assignees: John Grant COLLINS, Canberra (AU); JAMES COOK UNIVERSITY, Townsville (AU)

(21) Appl. No.: 13/334,741

(22) Filed: Dec. 22, 2011

(51) Int. Cl. A61K 31/555; A61P 31/04; C07F 15/00

(52) U.S. Cl. 514/188; 546/10; 546/2

(57) ABSTRACT

The present invention relates to ruthenium complexes, and in particular di- and multi-nuclear ruthenium complexes which may be used as antimicrobial agents. The invention also relates to pharmaceutical compositions comprising such complexes, and methods for their use in treating or preventing microbial infections.

### FIELD OF THE INVENTION

The present invention relates to ruthenium complexes, and in particular di- and multi-nuclear ruthenium complexes which may be used as antimicrobial agents. The invention also relates to pharmaceutical compositions comprising such complexes, and methods for their use in treating or preventing microbial infections.

### BACKGROUND OF THE INVENTION

The development of antimicrobial agents has been one of the major advances in medical science. However, a consequence of their widespread use has been the development of drug-resistant populations of bacteria. Infection by these organisms is emerging as an important cause of morbidity and mortality worldwide. In a recent update from the Infectious Diseases Society of America, Enterococcus faecium, Staphylococcus aureus (S. aureus), Klebsiella pneumoniae, Acinetobacter baumanii, Pseudomonas aeruginosa (P. aeruginosa) and Enterobacter species were identified as the pathogens of most current concern. In particular, methicillin-resistant S. aureus (MRSA), fluoroquinolone-resistant P. aeruginosa and vancomycin-resistant Enterococcus (VRE) show rapidly increasing rates of infection. There is clearly a need for the development of new antimicrobial agents, but perhaps more importantly, there is the need for the development of new classes of antimicrobials that may not be as susceptible to the bacterial mechanisms of resistance developed against the current range of drugs.

### SUMMARY OF THE INVENTION

It has now been found that a particular class of ruthenium complexes having two or more ruthenium centres is effective in the treatment and/or prevention of microbial infections.

Accordingly in one aspect the present invention provides a compound of the following formula:

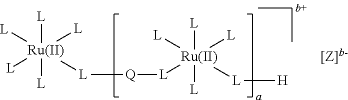

wherein:
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—;
wherein when the compound does not contain a labile ligand and a=1 then Q contains at least one —NH—, —N(alkyl)- or —O— group.

In another aspect the present invention provides a pharmaceutical composition comprising a compound of the following formula:

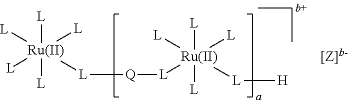

wherein:
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—;
wherein when the compound does not contain a labile ligand and a=1 then Q contains at least one —NH—, —N(alkyl)- or —O— group;
or a pharmaceutically acceptable salt thereof together with at least one pharmaceutically acceptable carrier or diluent.

In another aspect the present invention provides a method of preventing or treating a microbial infection comprising administering to a subject in need thereof an effective amount of a compound of the following formula:

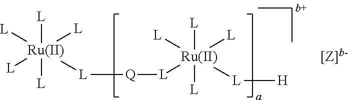

wherein:
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—.

In another aspect the present invention provides a compound of the following formula:

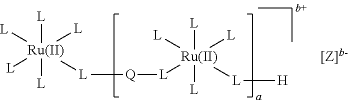

wherein:
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—;
for use in preventing or treating a microbial infection.

In another aspect the present invention provides use of a compound of the following formula:

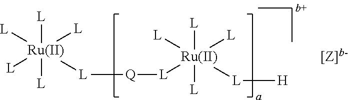

wherein:
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—; in the manufacture of a medicament for preventing or treating a microbial infection.

These compounds and uses thereof have been shown to be active against a range of organisms, including antibiotic resistant bacteria.

In some embodiments of the invention one or more of the following definitions apply:

a is an integer from 1 to 3, preferably a is 1;
Q is an alkylene linking group wherein any one or more methylene moieties in alkylene is optionally independently replaced with —NH—, —N(alkyl)- or —O—, preferably Q is a C₂₋₁₆alkylene linking group wherein any one or more methylene moieties in alkylene is optionally independently replaced with —NH—, —N(alkyl)- or —O—, more preferably Q is a C₂₋₁₆alkylene linking group;
b is an integer from 2 to 8, typically when a is 1 then b is an integer from 2 to 4, preferably when a is 1 then b is 2 or 3, more preferably when a is 1 then b is 3, typically when a is 2 then b is an integer from 3 to 6, preferably when a is 2 then b is an integer from 3 to 5, more preferably when a is 2 then b is 4 or 5 such as when one terminal ruthenium centre does not complex a labile ligand, typically when a is 3 then b is an integer from 4 to 8, preferably when a is 3 then b is an integer from 4 to 7, more preferably when a is 3 then b is an integer from 5 to 7 such as when one terminal ruthenium centre does not complex a labile ligand;
Z represents one or more counteranions, preferably halide (such as fluoride, chloride, bromide or iodide), acetate, succinate, maleate, trifluoromethanesulfonate (triflate) or hexafluorophosphate and mixtures thereof; and
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom, preferably pyridyl is selected from optionally substituted bipyridine (bipy or bpy), optionally substituted terpyridine (terpy) and optionally substituted phenanthroline (phen), such as methylbipyridine, terpyridine, phenanthroline and tetramethylphenanthroline, preferably labile ligand is selected from halide (such as iodide, bromide and chloride, preferably chloride) and water.

### BRIEF DESCRIPTION OF THE DRAWINGS

FIG. 1 depicts the results of 24 h haemolysis assays for ΔΔ-Rubb₇, ΔΔ-Rubb₁₀, ΔΔ-Rubb₁₂ and ΔΔ-Rubb₁₆ with freshly-collected human red blood cells, fitted as logarithmic curves.

FIG. 2 depicts haemolytic dose-response curves of ΔΔ-Rubb₁₆ (a) and ΔΔ-Rubb₁₂ (b) for different incubation times (2, 4, 8 and 24 h).

FIG. 3 depicts a comparison of the antimicrobial activity, haemolytic activity and cytotoxicity against the THP-1 cell line after 24 h incubation with ΔΔ-Rubb₇, ΔΔ-Rubb₁₀, ΔΔ-Rubb₁₂ and ΔΔ-Rubb₁₆. The ratio of HC₅₀ and IC₅₀ of the complexes to their own MIC is shown.

### DETAILED DESCRIPTION OF THE INVENTION

As defined herein, each ligand (L) that complexes the ruthenium centres of the compounds of the invention is selected from pyridyl ligand or labile ligand:

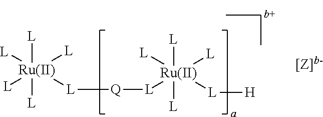

In some embodiments the compounds of the invention are symmetric with respect to the types of ligands on the ruthenium centres, such as [{Ru(phen)₂}₂(μ-(bpy(Me)CH₂CH₂bpy(Me)))](PF₆)₄ where each ruthenium centre complexes the same group of pyridyl ligands, and [{Ru(terpy)Cl}₂(μ-(bpy(Me)CH₂CH₂bpy(Me)))]Cl₂ where each ruthenium centre complexes the same group of pyridyl and labile ligands (chloride).

In some embodiments the complexes are non-symmetric with respect to the types of ligands on the ruthenium centres, such as [{Ru(phen)₂}(μ-(bpy(Me)CH₂CH₂ bpy(Me))) {Ru(phen)(bpy)}]Cl₄ where each ruthenium centre complexes a different group of pyridyl ligands and [{Ru(terpy)Cl}(μ-(bpy(Me)CH₂CH₂ bpy(Me))){Ru(phen)(Me₂bpy)}]Cl₃ where each ruthenium centre complexes a different group of pyridyl and/or labile ligands (chloride). In this latter example, one ruthenium centre complexes a labile ligand and the other ruthenium centre does not complex a labile ligand. Such non-symmetric compounds wherein one terminal ruthenium centre complexes a labile ligand and one terminal ruthenium centre does not complex a labile ligand are believed to be particularly advantageous as compounds of the invention and in the prevention or treatment of a microbial infection as defined herein. Accordingly in one aspect the present invention provides a compound of the following formula:

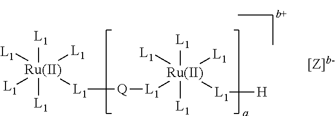

wherein:
a is an integer from 1 to 3, wherein when a is greater than 1 each Q may be the same or different;
b is an integer from 2 to 8;
Z represents one or more counteranions;
each L₁ may be the same or different and is independently selected from pyridyl ligand such that each pyridyl ligand forms a polydentate ligand together with one or more other pyridyl ligands on the same Ru(II) atom;
L₂ is a labile ligand; and
Q is an alkylene linking group wherein any one or more methylene moieties in alkylene is optionally independently replaced with —NH—, —N(alkyl)- or —O—.

The compound may be used in the prevention or treatment of a microbial infection.

As used herein the term “pyridyl ligand” takes its standard meaning in the art and refers to the class of ligands which comprise one or more pyridyl groups (such as derivatives of pyridine) as well as pyridine itself. Typically complexation of the ligand to the ruthenium nucleus in the compounds of the invention occurs though the nitrogen atom within the or each pyridine ring. The pyridyl ligands of the present invention form a polydentate ligand together with one or more other pyridyl ligands on the same ruthenium atom. In this respect the pyridyl ligands may be referred to as polypyridyl ligands. As used herein the term “polydentate ligand” takes its standard meaning in the art and refers to ligands which may be bidentate (or didentate), tridentate, tetradentate, etc. The polypyridyl ligands may be optionally substituted with suitable groups including alkyl groups such as methyl groups. Examples of optionally substituted polypyridyl ligands according to the invention include optionally substituted bipyridine (bipy or bpy), optionally substituted terpyridine (terpy) and optionally substituted phenanthroline (phen), such as methylbipyridine, terpyridine, phenanthroline and tetramethylphenanthroline. By way of example, in the compound represented by the following structure:

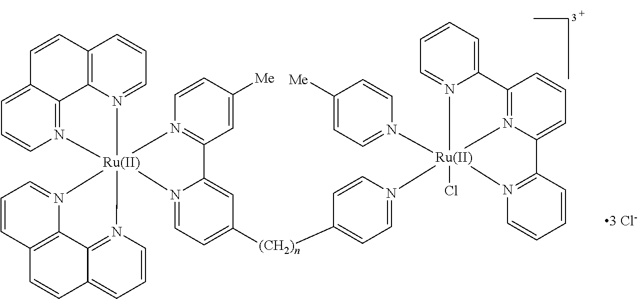

the skilled worker shall appreciate that the ruthenium centre on the left of the structure coordinates two phenanthroline ligands and one substituted bipyridine ligand, whereas the ruthenium centre on the right of the structure coordinates one terpyridine ligand, one substituted bipyridine ligand and one chloride ligand.

As used herein the term “labile ligand” takes its standard meaning in the art and refers to the class of ligands which may readily dissociate from the ruthenium centre to which the ligand is complexed. The labile ligand may be charged or uncharged. Examples of labile ligands according to the invention are halide (such as iodide, bromide and chloride, preferably chloride) and water. By way of example, in the compound represented by the following structure:

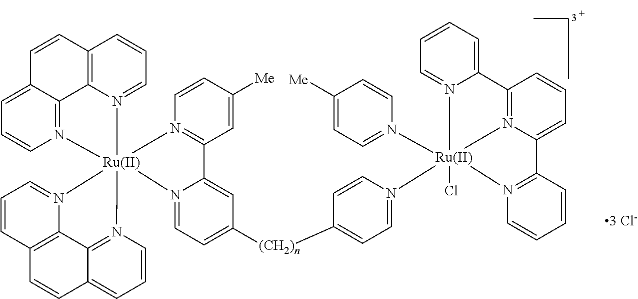

the skilled worker shall appreciate that the ruthenium atom on the right of the structure coordinates a labile chloride ligand. When contacted with water, under suitable conditions for a sufficient time, a water molecule may substitute for the labile chloride ligand. In this event the overall charge of the cationic portion of the compound shall be increased to 4⁺ thereby leading to an association with four chloride counteranions as shown below: 4+

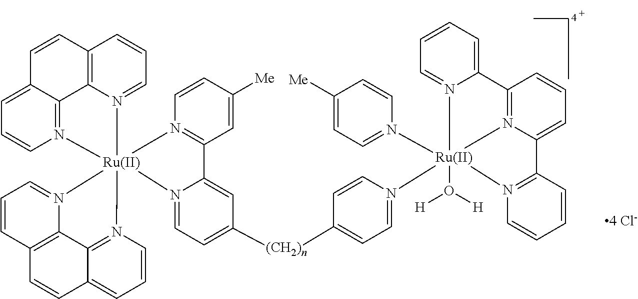

Compounds of the invention bearing at least one labile ligand, and uses of those compounds in the prevention and treatment of microbial infection, are in some circumstances preferred. Without wishing to be bound by theory, it is believed that the labile ligand typically dissociates from the ruthenium nucleus under physiological conditions, which may occur in the presence of other ligands such as water, or heteroatoms present in nucleic acids (eg DNA, RNA) or proteins. In some proposed mechanisms, the labile ligand possibly dissociates through stepwise combinations of ligands such as substitution with water followed by substitution with a phosphate group of a nucleic acid followed by substitution with N7 of a nucleic acid (eg GMP).

In one aspect the present invention provides a compound of the following formula:

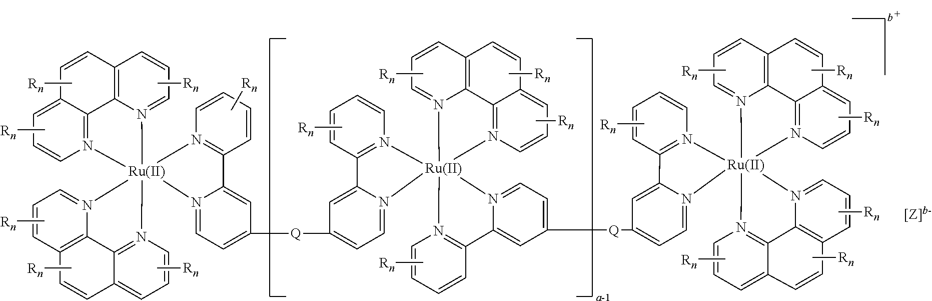

wherein:
a is an integer from 1 to 3, wherein when a is greater than 1 each Q may be the same or different;
b is an integer from 4 to 8;
Z represents one or more counteranions;
Q is an alkylene linking group wherein any one or more methylene moieties in alkylene is optionally independently replaced with —NH—, —N(alkyl)- or —O—;
each R may be the same or different and is independently selected from an alkyl group, such as methyl;
each n is independently selected from 0, 1 or 2.

The compound may be used in the prevention or treatment of a microbial infection.

In some embodiments, when a=1 then Q contains at least one —NH—, —N(alkyl)- or —O— group. In further embodiments when a=1 then Q is not selected from 1,2-ethylene, 1,5-pentylene, 1,7-heptylene, 1,10-decylene, 1,12-dodecylene, 1,14-tetradecylene and 1,16-hexadecylene, or any one of:

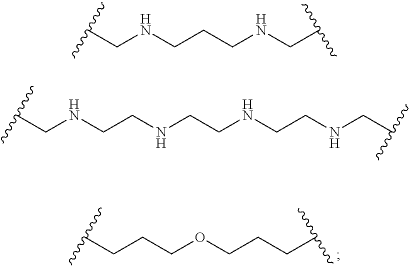 [SMILES: *CNCCCNC* ; *CNCCNCCNCCNC* ; *CCCOCCC*]

and when a=2 or 3 then Q is not selected from 1,7-heptylene. In still further embodiments when a=1, 2 or 3 then Q is not selected from 1,2-ethylene, 1,5-pentylene, 1,7-heptylene, 1,10-decylene, 1,12-dodecylene, 1,14-tetradecylene and 1,16-hexadecylene, or any one of:

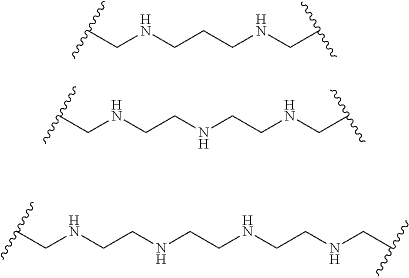 [SMILES: *CNCCCNC* ; *CNCCNCCNC* ; *CNCCNCCNCCNC*]

or any one of:

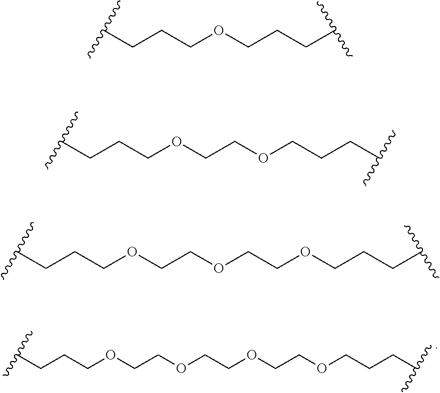 [SMILES: *CCCOCCC* ; *CCCOCCOCCC* ; *CCCOCCOCCOCCC* ; *CCCOCCOCCOCCOCCC*]

In another aspect the present invention provides a compound of the following formula:

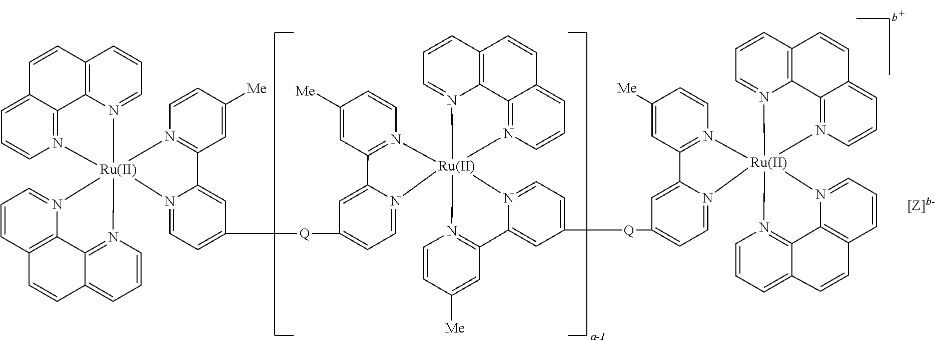

wherein:
a is an integer from 1 to 3, wherein when a is greater than 1 each Q may be the same or different;
b is an integer from 4 to 8;
Z represents one or more counteranions;
Q is an alkylene linking group wherein any one or more methylene moieties in alkylene is optionally independently replaced with —NH—, —N(alkyl)- or —O—.

The compound may be used in the prevention or treatment of a microbial infection.

In some embodiments, when a=1 then Q contains at least one —NH—, —N(alkyl)- or —O— group. In further embodiments when a=1 then Q is not selected from 1,2-ethylene, 1,5-pentylene, 1,7-heptylene, 1,10-decylene, 1,12-dodecylene, 1,14-tetradecylene and 1,16-hexadecylene, or any one of:

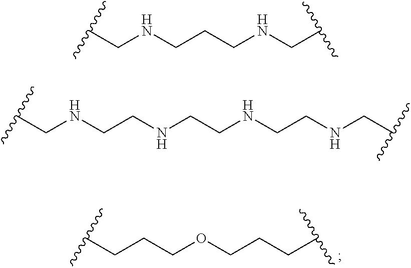 [SMILES: *CNCCCNC* ; *CNCCNCCNCCNC* ; *CCCOCCC*]

and when a=2 or 3 then Q is not selected from 1,7-heptylene. In still further embodiments when a=1, 2 or 3 then Q is not selected from 1,2-ethylene, 1,5-pentylene, 1,7-heptylene, 1,10-decylene, 1,12-dodecylene, 1,14-tetradecylene and 1,16-hexadecylene, or any one of:

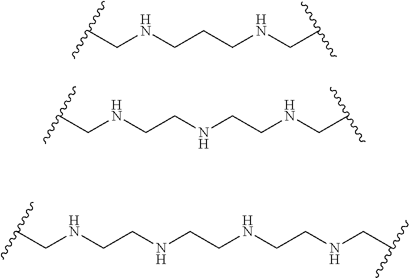 [SMILES: *CNCCCNC* ; *CNCCNCCNC* ; *CNCCNCCNCCNC*]

or any one of:

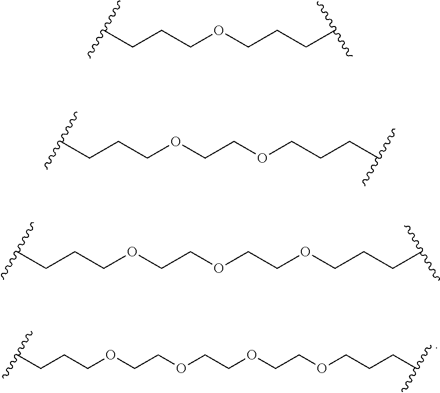 [SMILES: *CCCOCCC* ; *CCCOCCOCCC* ; *CCCOCCOCCOCCC* ; *CCCOCCOCCOCCOCCC*]

In one aspect the present invention provides a compound of the following formula:

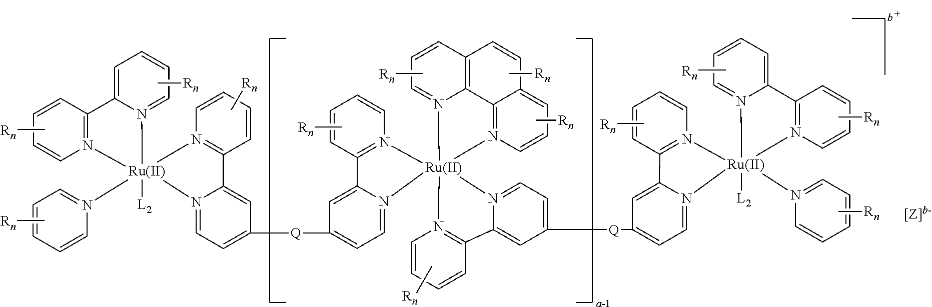

wherein:
a is an integer from 1 to 3, wherein when a is greater than 1 each Q may be the same or different;
each L₂ may be the same of different and is independently selected from a labile ligand;
b is an integer from 2 to 8;
Z represents one or more counteranions;
Q is an alkylene linking group wherein any one or more methylene moieties in alkylene is optionally independently replaced with —NH—, —N(alkyl)- or —O—;
each R may be the same or different and is independently selected from an alkyl group, such as methyl;
each n is independently selected from 0, 1 or 2.

The compound may be used in the prevention or treatment of a microbial infection.

In some embodiments, when a=1 then Q contains at least one —NH—, —N(alkyl)- or —O— group. In further embodiments when a=1 then Q is not selected from 1,7-heptylene, 1,10-decylene, 1,12-dodecylene and 1,14-tetradecylene.

In another aspect the present invention provides a compound of the following formula:

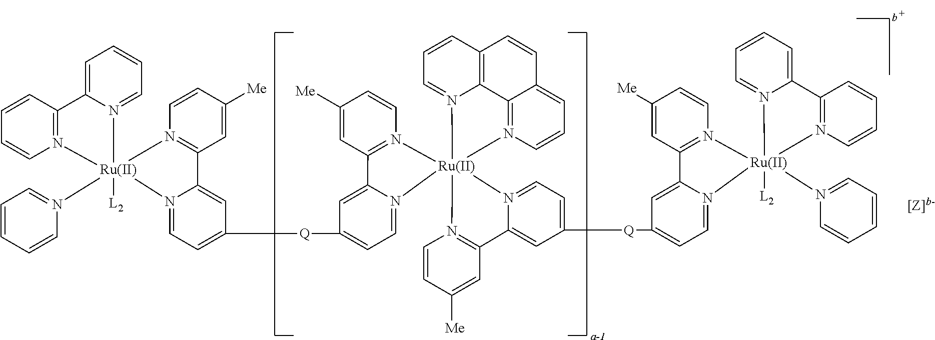

wherein:
a is an integer from 1 to 3, wherein when a is greater than 1 each Q may be the same or different;
each L₂ may be the same of different and is independently selected from a labile ligand;
b is an integer from 2 to 8;
Z represents one or more counteranions; and
Q is an alkylene linking group wherein any one or more methylene moieties in alkylene is optionally independently replaced with —NH—, —N(alkyl)- or —O—.

The compound may be used in the prevention or treatment of a microbial infection.

In further embodiments when a=1 then Q is not selected from 1,7-heptylene, 1,10-decylene, 1,12-dodecylene and 1,14-tetradecylene.

In one aspect the present invention provides a compound of the following formula:

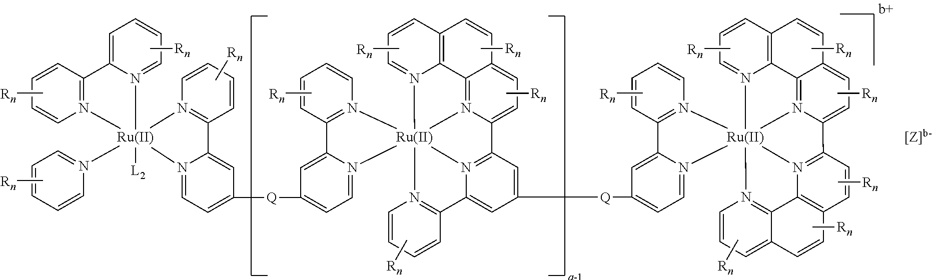

wherein:
a is an integer from 1 to 3, wherein when a is greater than 1 each Q may be the same or different;
each L₂ may be the same of different and is independently selected from a labile ligand;
b is an integer from 2 to 8;
Z represents one or more counteranions;
Q is an alkylene linking group wherein any one or more methylene moieties in alkylene is optionally independently replaced with —NH—, —N(alkyl)- or —O—;
each R may be the same or different and is independently selected from an alkyl group, such as methyl;
each n is independently selected from 0, 1 or 2.

The compound may be used in the prevention or treatment of a microbial infection.

In another aspect the present invention provides a compound of the following formula:

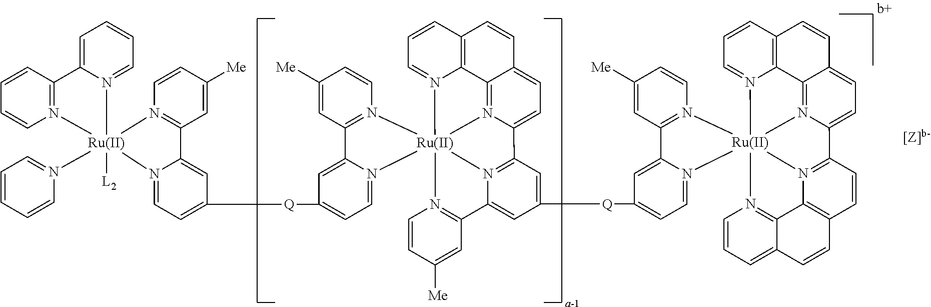

wherein:
a is an integer from 1 to 3, wherein when a is greater than 1 each Q may be the same or different;
L₂ is a labile ligand;
b is an integer from 2 to 8;
Z represents one or more counteranions; and
Q is an alkylene linking group wherein any one or more methylene moieties in alkylene is optionally independently replaced with —NH—, —N(alkyl)- or —O—.

The compound may be used in the prevention or treatment of a microbial infection.

The skilled person will recognise that a number of atoms, such as the complexed ruthenium centres, within the compounds of the invention may exist in more than one stereoisomeric form. The present invention contemplates within its scope compounds of all possible absolute configurations about all such atoms. For example, the compounds of the invention may exist as mixtures of Λ- and Δ-stereoisomeric forms about any one or more of the ruthenium centres, including racemic mixtures or enantioenriched mixtures, or may exist in enantiopure form wherein each ruthenium centre exists as the Λ- or the Δ-stereoisomeric form. In some embodiments, each ruthenium centre within the compound has the same absolute configuration such that for a compound bearing two ruthenium centres the stereochemical configuration is either ΛΛ- or ΔΔ-. In those compounds bearing more than two ruthenium centres, the non-terminal ruthenium centres may exist as racemic mixtures whereas the terminal ruthenium centres may be in either the Λ- or Δ-stereoisomeric form.

The skilled person will appreciate that there are a range of techniques available to produce the compounds of the invention in racemic, enantioenriched or enantiopure forms. For example, enantioenriched or enantiopure forms of the compounds may be produced through stereoselective synthesis and/or through the use of chromatographic or selective recrystallisation techniques.

Accordingly in some embodiments the present invention provides a compound of the formula:

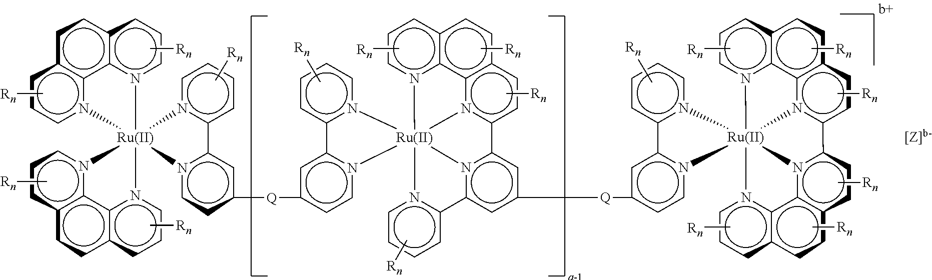

wherein:
a is an integer from 1 to 3, wherein when a is greater than 1 each Q may be the same or different;
b is an integer from 4 to 8;
Z represents one or more counteranions;
Q is an alkylene linking group wherein any one or more methylene moieties in alkylene is optionally independently replaced with —NH—, —N(alkyl)- or —O—;
each R may be the same or different and is independently selected from an alkyl group, such as methyl;
each n is independently selected from 0, 1 or 2.

The compound may be used in the prevention or treatment of a microbial infection.

In some embodiments, when a=1 then Q contains at least one —NH—, —N(alkyl)- or —O— group. In further embodiments when a=1 then Q is not selected from 1,2-ethylene, 1,5-pentylene, 1,7-heptylene, 1,10-decylene, 1,12-dodecylene, 1,14-tetradecylene and 1,16-hexadecylene, or any one of:

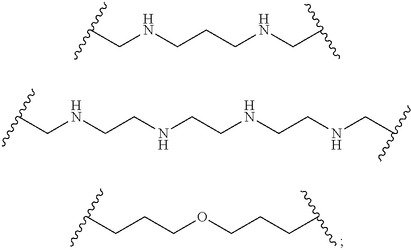 [SMILES: *CNCCCNC* ; *CNCCNCCNCCNC* ; *CCCOCCC*]

and when a=2 or 3 then Q is not selected from 1,7-heptylene. In still further embodiments when a=1, 2 or 3 then Q is not selected from 1,2-ethylene, 1,5-pentylene, 1,7-heptylene, 1,10-decylene, 1,12-dodecylene, 1,14-tetradecylene and 1,16-hexadecylene,

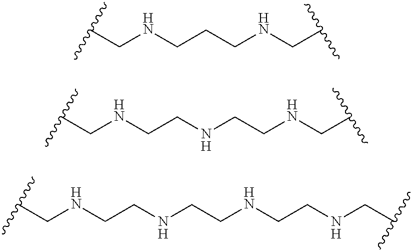 [SMILES: *CNCCCNC* ; *CNCCNCCNC* ; *CNCCNCCNCCNC*]

or any one of:

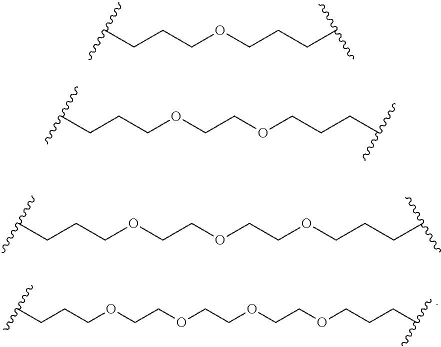 [SMILES: *CCCOCCC* ; *CCCOCCOCCC* ; *CCCOCCOCCOCCC* ; *CCCOCCOCCOCCOCCC*]

or any one of:

In some embodiments the compounds of the present invention have the following formula:

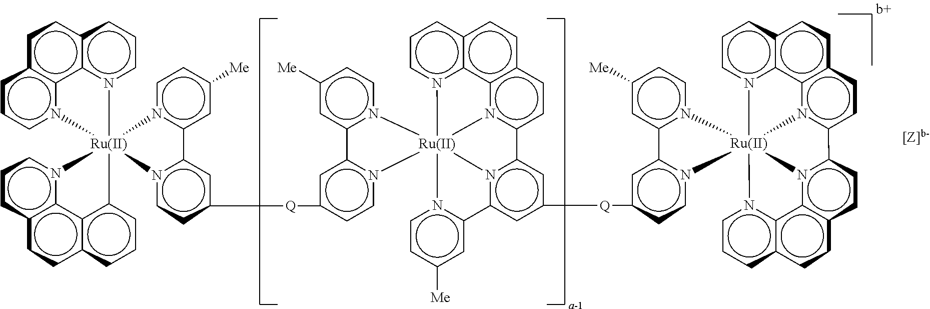

wherein:
a is an integer from 1 to 3, wherein when a is greater than 1 each Q may be the same or different;
b is an integer from 4 to 8;
Z represents one or more counteranions;
Q is an alkylene linking group wherein any one or more methylene moieties in alkylene is optionally independently replaced with —NH—, —N(alkyl)- or —O—.

The compound may be used in the prevention or treatment of a microbial infection.

In some embodiments, when a=1 then Q contains at least one —NH—, —N(alkyl)- or —O— group. In further embodiments when a=1 then Q is not selected from 1,2-ethylene, 1,5-pentylene, 1,7-heptylene, 1,10-decylene, 1,12-dodecylene, 1,14-tetradecylene and 1,16-hexadecylene, or any one of:

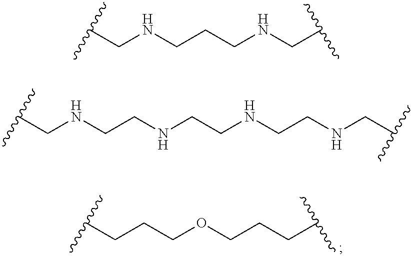 [SMILES: *CNCCCNC* ; *CNCCNCCNCCNC* ; *CCCOCCC*]

and when a=2 or 3 then Q is not selected from 1,7-heptylene. In still further embodiments when a=1, 2 or 3 then Q is not selected from 1,2-ethylene, 1,5-pentylene, 1,7-heptylene, 1,10-decylene, 1,12-dodecylene, 1,14-tetradecylene and 1,16-hexadecylene, or any one of:

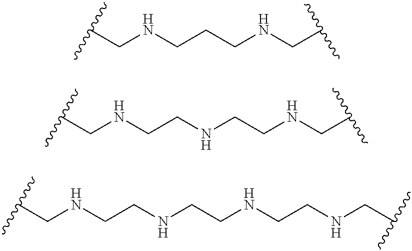 [SMILES: *CNCCCNC* ; *CNCCNCCNC* ; *CNCCNCCNCCNC*]

or any one of:

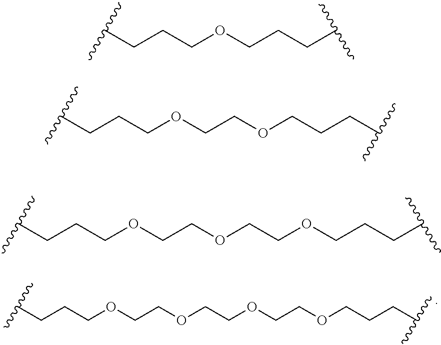 [SMILES: *CCCOCCC* ; *CCCOCCOCCC* ; *CCCOCCOCCOCCC* ; *CCCOCCOCCOCCOCCC*]

In some embodiments a is 2 and the compound has the stereochemistry shown below:

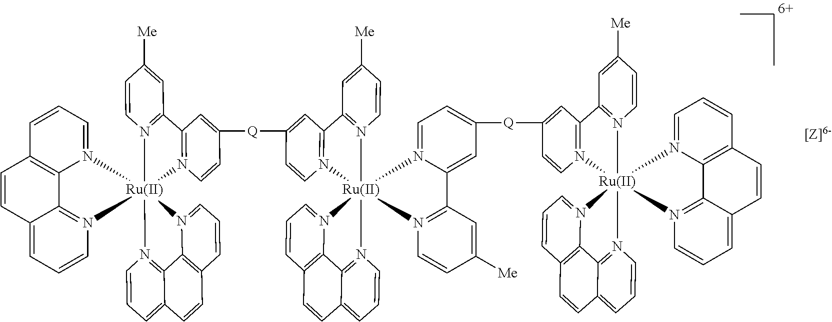

wherein:
each Q may be the same or different and is an alkylene linking group wherein any one or more methylene moieties in alkylene is optionally independently replaced with —NH—, N(alkyl)- or —O—; and
Z represents one or more counteranions.

The compound may be used in the prevention or treatment of a microbial infection.

In some embodiments Q contains at least one —NH—, —N(alkyl)- or —O— group. In further embodiments Q is not selected from 1,7-heptylene.

In some embodiments a is 3 and the compound has the stereochemistry shown below:

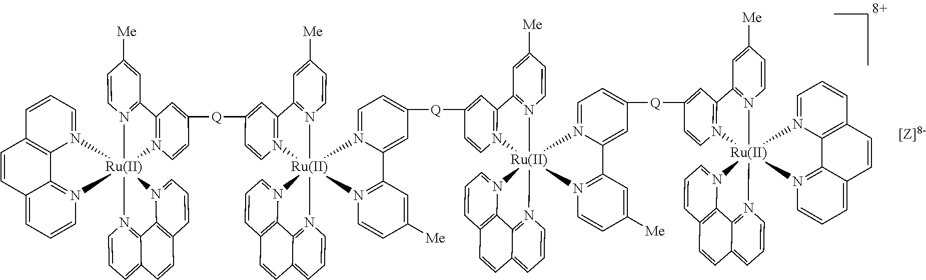

wherein each Q may be the same or different and is an alkylene linking group wherein any one or more methylene moieties in alkylene is optionally independently replaced with —NH—, —N(alkyl)- or —O—; and
Z represents one or more counteranions

The compound may be used in prevention or treatment of a microbial infection.

In some embodiments Q contains at least one —NH—, —N(alkyl)- or —O— group. In further embodiments Q is not selected from 1,7-heptylene.

As used herein, the term “alkylene” is intended to denote the divalent form of “alkyl” as herein defined. The term “alkyl” denotes straight chain, branched or cyclic alkyl, for example C₁₋₄₀₀ alkyl, or C₁₋₂₀ or C₂₋₁₆ Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- or 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1,2-pentylheptyl, tridecyl, tetradecyl, pentadecyl, hexadecyl, heptadecyl, octadecyl, nonoadecyl, eicosyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like.

In preferred embodiments the alkylene linking group “Q” is a flexible alkylene linking group. Examples of flexible alkylene linking group include linear alkylene groups, such as methylene, ethylene, propylene, butylene, pentylene, hexylene, heptylene, octylene, nonylene, decylene, undecylene, dodecylene, tridecylene, tetradecylene, pentadecylene, and hexdecylene. Preferably the alkylene linking group links through the terminal carbon atoms of the group, namely through the α,ω-carbon atoms of the group. Specific examples include 1,2-ethylene, 1,5-pentylene, 1,7-heptylene, 1,10-decylene, 1,12-dodecylene, 1,14-tetradecylene and 1,16-hexadecylene as shown below:

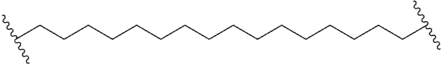 [SMILES: *CCCCCCCCCCCCCCCC*]

As herein defined Q is an alkylene linking group wherein any one or more methylene moieties in alkylene is optionally independently replaced with —NH—, —N(alkyl)- or —O—, such as —NH— or —O—. It will be understood that the replacement of a methylene group in an alkylene group with —NH— or —N(alkyl)- (such as —NMc-, —N(ethyl)-, —N(propyl)-, etc) will create an amine. For example, replacement of a methylene group in 1,3-propylene with —NH— will create a methaminomethyl linking group as shown below:

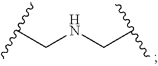 [SMILES: *CNC*]

and replacement of a methylene group in 1,3-propylene with —NMe- will create a methamino(methyl)methyl linking group as shown below:

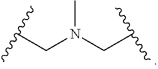 [SMILES: *CN(C)C*]

Likewise it will be understood that the replacement of a methylene group in an alkylene group with —O— will create an ether. For example, replacement of a methylene group in 1,3-propylene with —O— will create a methoxymethyl linking group as shown below:

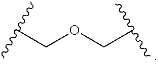 [SMILES: *COC*]

In some embodiments, more than one methylene group in the alkylene linking group, Q, will be independently replaced with —NH—, —N(alkyl)- or —O—. Whilst mixed ether-amine linking groups are contemplated, typically the linking group will be either an alkylene, a (poly)aminoalkylene or a (poly)oxyalkylene.

Examples of polyaminoalkylene linking groups are provided below:

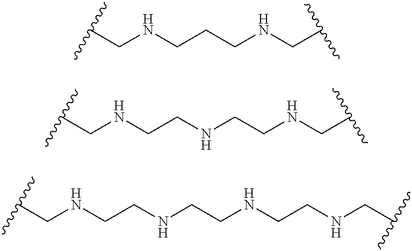 [SMILES: *CNCCCNC* ; *CNCCNCCNC* ; *CNCCNCCNCCNC*]

Examples of (poly)oxyalkylene linking groups are provided below:

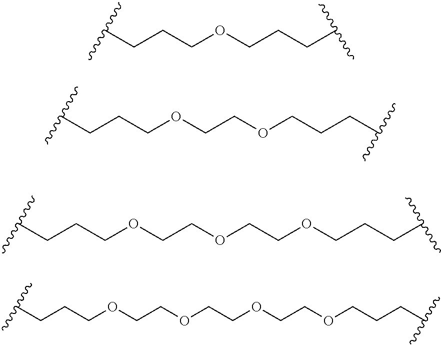 [SMILES: *CCCOCCC* ; *CCCOCCOCCC* ; *CCCOCCOCCOCCC* ; *CCCOCCOCCOCCOCCC*]

It will be understood that the linking group may be used to alter the lipophilicity, flexibility and size of the ruthenium complexes of the present invention. For example, the skilled person will recognise that under certain conditions the polyaminoalkylene linking groups shown above will become protonated. In some embodiments this protonation may be preferable to aid in water solubility, or in other embodiments it may be deleterious if increased lipophilicity is desired. The skilled worker is therefore provided with a useful handle to alter the lipophilicity of the complexes of the present invention. One such way in which the lipophilicity of the complexes of the present invention may be altered is by making a pharmaceutically acceptable salt of an amine group in Q.

As used herein the term “counteranion” refers to any negatively charged group, such as organic or inorganic anionic groups, which renders the overall charge of the compounds of the invention neutral. In particular, the entity [Z]^{b-} refers to one or more counteranions providing an overall negative charge “b−” which is sufficient to render the charge of the compounds of the invention neutral. Counteranions may be atomic, such as halide including fluoride, chloride, bromide and iodide counteranions. Counteranions can also be molecular, such as acetate, succinate, maleate, trifluoromethanesulfonate (tritlate) and hexafluorophosphate. Counteranions can have a charge greater than 1, such as 2 or more. By way of example, in the compound represented by the following structure:

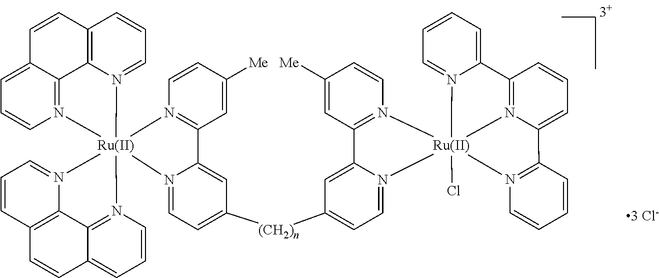

the skilled worker shall appreciate that the entity [Z]^{b-} referred to herein corresponds to the three chloride counteranions such that b is equal to 3. In preferred embodiments the counteranion is pharmaceutically acceptable.

The compounds and methods of the present invention may be used in the treatment and/or prevention of a range of microbial infections. As used herein, treatment may include alleviating or ameliorating the symptoms, diseases or conditions associated with the microbial infection being treated, including reducing the severity and/or frequency of the microbial infection. As used herein, prevention may include preventing or delaying the onset of, inhibiting the progression of, or halting or reversing altogether the onset or progression of the particular symptoms, disease or condition associated with a microbial infection.

The terms “microbial”, “microorganism”, etc includes any microscopic organism or taxonomically related macroscopic organism within the categories algae, bacteria, fungi, yeast and protozoa or the like.

The bacterial infection may be caused by one or more species selected from one or more of the Gram-negative bacterial genera: Acinetobacter; Actinobacillus; Bartonella; Bordetella; Brucella; Burkholderia; Campylobacter; Cyanobacteria; Enterobacter; Erwinia; Escherichia; Francisella; Helicobacter; Hemophilus; Klebsiella; Legionella; Moraxella; Morganella; Neisseria; Pasteurella; Proteus; Providencia; Pseudomonas; Salmonella; Serratia; Shigella; Stenotrophomonas; Treponema; Vibrio; and Yersinia. Specific examples include, but are not limited to, infections caused by Bacteroides, Bordetella pertussis, Brucella, Campylobacter infections, enterohaemorrhagic Escherichia coli (EHEC) enteroinvasive Escherichia coli (EIEC), enterotoxigenic Escherichia coli (ETEC), Haemophilus influenzae, Helicobacter pylori, Klebsiella pneumoniae, Legionella spp., Moraxella catarrhalis, Neisseria gonnorrhoeae, Neisseria meningitidis, Proteus spp., Pseudomonas aeruginosa, Salmonella spp., Shigella spp., Vibrio cholera and Yersinia; acid fast bacteria including Mycobacterium tuberculosis, Mycobacterium avium-intracellulare, Myobacterium johnei, Mycobacterium leprae, atypical bacteria, Chlamydia, Mycoplasma, Rickettsia, Spirochetes, Treponema pallidum, Borrelia recurrentis, Borrelia burgdorfii and Leptospira icterohemorrhagiae and other miscellaneous bacteria, including Actinomyces and Nocardia.

The bacterial infection may be caused by one or more species selected from one or more of the Gram-positive bacterial genera: Actinobacteria; Bacillus; Clostridium; Corynebacterium; Enterococcus; Listeria; Nocardia; Staphylococcus; and Streptococcus. Specific examples include, but are not limited to, infections caused by Bacillus cereus, Bacillus anthracis, Clostridium botulinum, Clostridium difficile, Clostridium tetani, Clostridium perfringens, Corynebacteria diphtheriae, Enierococcus (Streptococcus D), Listeria monocytogenes, Pneumoccoccal infections (Streptococcus pneumoniae), Staphylococcal infections and Streptococcal infections.

Fungal infections include, but are not limited to, infections caused by Alternaria alternata, Aspergillus flavus, Aspergillus fumigatus, Aspergillus nidulans, Aspergillus niger, Aspergillus versicolor, Blastomyces dermatiditis, Candida albicans, Candida dubliensis, Candida krusei, Candida parapsilosis, Candida tropicalis, Candida glabrata, Coccidioides immitis, Cryptococcus neoformans, Epidermophyton floccosum, Histoplasma capsulatum, Malassezia furfur, Microsporum canis, Mu cor spp., Paracoccidioides brasiliensis, Penicillium marneffei, Pityrosporum ovale, Pneumocystis carinii, Sporothrix schenkii, Trichophyton rubrum, Trichophyton interdigitale, Trichosporon beigelii and Rhodotorula spp.

Yeast infections include, but are not limited to, infections caused by Brettanonyces clausenii, Brettanomyces custerii, Brettanomyces anomalous, Brettanomyces naardenensis, Candida himilis, Candida intermedia, Candida saki, Candida solani, Candida tropicalis, Candida versatilis, Candida bechii, Candida famata, Candida lipolytica, Candida stellata, Candida vini, Debaromyces hansenii, Dekkera intermedia, Dekkera bruxellensis, Geotrichium sandidum, Hansenula fabiani, Hanseniaspora uvarum, Hansenula anomala, Hanseniaspora guillermondii, Hanseniaspora vinae, Kluyveromyces lactis, Kloekera apiculata, Kluveromyces marxianus, Kluyveromyces fragilis, Metschikowia pulcherrima, Pichia guilliermodii, Pichia orientalis, Pichia fermenans, Pichia memranefaciens, Rhodotorula Saccharomyces bayanus, Saccharomyces cerevisiae, Saccharomyces dairiensis, Saccharomyces exigus, Saccharomyces uinsporus, Saccharomyces uvarum, Saccharomyces oleaginosus, Saccharomyces boulardii, Saccharomycodies ludwigii, Schizosaccharomyces ponzbe, Torulaspora delbruekii, Torulopsis stellata, Zygoaccharomyces bailli and Zvgosaccharomyces rouxii.

Protozoal infections include, but are not limited to, infections caused by Leishmania, Toxoplasma, Plasmodia (which are understood to be the causative agent(s) of malarial infection), Theileria, Anaplasma, Giardia, Trichomonas, Trypanosoma, Coccidia, and Babesia. Specific examples include Trypanosoma cruzi, Eimeria tenella, Plasmodium falciparum, Plasmodium vivax, Plasmodium malariae, Plasmodium knowlesi or Plasmodium ovale.

Preferably, the microbial infection is caused by either a Gram-positive or a Gram-negative bacterium, for example, Staphylococcus aureus (including MRSA), Enterococcus fecalis, Escherichia coli, Klebsiella pneumonia, Salmonella typhimurium or pseudotuberculosis, Acinetobacter, Pseudomonas aeruginosa, Clostridium perfringens, Clostridium dijficile, Campylobacter jejuni or Bacteroides fragilis; a fungal or yeast infection, for example, Trichophyton interdigitale; Aspergillus fumigatus or Candida albicans; or a protozoal infection, for example Plasmodium falciparum.

Examples of microbial infections include bacterial or fungal wound infections, mucosal infections, enteric infections, septic conditions, pneumonia, trachoma, ornithosis, trichomoniasis, fungal infections and salmonellosis, such as in veterinary practice. The compounds of the invention may also be used for the treatment of resistant microbial species or in various fields where antiseptic treatment or disinfection of materials is required, for example, surface disinfection.

The term “subject” as used herein refers to any animal having a disease or condition which requires treatment with a pharmaceutically-active agent. The subject may be a mammal, preferably a human, or may be a domestic or companion animal. While it is particularly contemplated that the compounds of the invention are suitable for use in medical treatment of humans, it is also applicable to veterinary treatment of animals.

The compounds of the invention may be in crystalline form or as solvates (e.g. hydrates) and it is intended that both forms are within the scope of the present invention. The term “solvate” is a complex of variable stoichiometry formed by a solute (in this invention, a compound of the invention) and a solvent. Such solvents should preferably not interfere with the biological activity of the solute. Solvents may be, by way of example, water, acetone, ethanol or acetic acid. Methods of solvation are generally known within the art.

The present invention also provides a pharmaceutical composition comprising a therapeutically effective amount of a compound as hereinbefore defined, or a pharmaceutically acceptable salt thereof, together with at least one pharmaceutically acceptable carrier or diluent.

Pharmaceutically acceptable acid addition salts may be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. For example, where the linking group represented by Q contains one or more amino groups, the amino groups may undergo reaction with an acid to form the acid addition salt.

Pharmaceutically acceptable base addition salts may be prepared from inorganic and organic bases. Corresponding counterions derived from inorganic bases include the sodium, potassium, lithium, ammonium, calcium and magnesium salts. Organic bases include primary, secondary and tertiary amines, substituted amines including naturally-occurring substituted amines, and cyclic amines, including isopropylamine, trimethyl amine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine.

Acid/base addition salts tend to be more soluble in aqueous solvents than the corresponding free acid/base forms.

The term “composition” is intended to include the formulation of an active ingredient with encapsulating material as carrier, to give a capsule in which the active ingredient (with or without other carrier) is surrounded by carriers.

While the compound as hereinbefore described, or pharmaceutically acceptable salt thereof, may be the sole active ingredient administered to the subject, the administration of other active ingredient(s) with the compound is within the scope of the invention. For example, the compound could be administered with one or more therapeutic agents in combination. The combination may allow for separate, sequential or simultaneous administration of the compound as hereinbefore described with the other active ingredient(s). The combination may be provided in the form of a pharmaceutical composition.

As will be readily appreciated by those skilled in the art, the route of administration and the nature of the pharmaceutically acceptable carrier will depend on the nature of the condition and the mammal to be treated. It is believed that the choice of a particular carrier or delivery system, and route of administration could be readily determined by a person skilled in the art. In the preparation of any formulation containing the compound care should be taken to ensure that the activity of the compound is not destroyed in the process and that the compound is able to reach its site of action without being destroyed. In some circumstances it may be necessary to protect the compound by means known in the art, such as, for example, micro encapsulation. Similarly the route of administration chosen should be such that the compound reaches its site of action.

Those skilled in the art may readily determine appropriate formulations for the compounds of the present invention using conventional approaches. Identification of preferred pH ranges and suitable excipients, for example antioxidants, is routine in the art. Buffer systems are routinely used to provide pH values of a desired range and include carboxylic acid buffers for example acetate, citrate, lactate and succinate. A variety of antioxidants are available for such formulations including phenolic compounds such as BHT or vitamin E, reducing agents such as methionine or sulphite, and metal chelators such as EDTA.

The compounds as hereinbefore described, or pharmaceutically acceptable salt thereof, may be prepared in parenteral dosage forms, including those suitable for intravenous, intrathecal, and intracerebral or epidural delivery. The pharmaceutical forms suitable for injectable use include sterile injectable solutions or dispersions, and sterile powders for the extemporaneous preparation of sterile injectable solutions. They should be stable under the conditions of manufacture and storage and may be preserved against reduction or oxidation and the contaminating action of microorganisms such as bacteria or fungi.

The solvent or dispersion medium for the injectable solution or dispersion may contain any of the conventional solvent or carrier systems for the compound, and may contain, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal and the like. In many cases, it will be preferable to include agents to adjust osmolarity, for example, sugars or sodium chloride. Preferably, the formulation for injection will be isotonic with blood. Prolonged absorption of the injectable compositions can be brought about by the use in the compositions of agents delaying absorption, for example, aluminium monostearate and gelatin. Pharmaceutical forms suitable for injectable use may be delivered by any appropriate route including intravenous, intramuscular, intracerebral, intrathecal, epidural injection or infusion.

Sterile injectable solutions are prepared by incorporating the active compound in the required amount in the appropriate solvent with various of the other ingredients such as those enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, preferred methods of preparation are vacuum drying or freeze-drying of a previously sterile-filtered solution of the active ingredient plus any additional desired ingredients.

Other pharmaceutical forms include oral and enteral formulations of the present invention, in which the active compound may be formulated with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal or sublingual tablets, troches, capsules, elixirs, suspensions, syrups, wafers, and the like. The amount of active compound in such therapeutically useful compositions is such that a suitable dosage will be obtained.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations, including those that allow specific delivery of the active compound to specific regions of the gut.

Liquid formulations may also be administered enterally via a stomach or oesophageal tube. Enteral formulations may be prepared in the form of suppositories by mixing with appropriate bases, such as emulsifying bases or water-soluble bases. It is also possible, but not necessary, for the compounds of the present invention to be administered topically, intranasally, intravaginally, intraocularly and the like.

The present invention also extends to any other forms suitable for administration, for example topical application such as creams, lotions and gels, or compositions suitable for inhalation or intranasal delivery, for example solutions, dry powders, suspensions or emulsions.

The compounds of the present invention may be administered by inhalation in the form of an aerosol spray from a pressurised dispenser or container, which contains a propellant such as carbon dioxide gas, dichlorodifluoromethane, nitrogen, propane or other suitable gas or combination of gases. The compounds may also be administered using a nebuliser.

Pharmaceutically acceptable vehicles and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate the compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically acceptable vehicle. The specification for the novel dosage unit forms of the invention are dictated by and directly dependent on (a) the unique characteristics of the active material and the particular therapeutic effect to be achieved, and (b) the limitations inherent in the art of compounding active materials for the treatment of disease in living subjects having a diseased condition in which bodily health is impaired as herein disclosed in detail.

As mentioned above the principal active ingredient may be compounded for convenient and effective administration in therapeutically effective amounts with a suitable pharmaceutically acceptable vehicle in dosage unit form. A unit dosage form can, for example, contain the principal active compound in amounts ranging from 0.25 μg to about 200 mg. Expressed in proportions, the active compound may be present in from about 0.25 μg to about 200 mg/mL of carrier. In the case of compositions containing supplementary active ingredients, the dosages are determined by reference to the usual dose and manner of administration of the said ingredients.

The terms “therapeutically effective amount” and “effective amount” refer to that amount which is sufficient to effect treatment, as defined below, when administered to an animal, preferably a mammal, more preferably a human in need of such treatment. The therapeutically effective amount or effective amount will vary depending on the subject and nature of bacterial infection being treated, the severity of the infection and the manner of administration, and may be determined routinely by one of ordinary skill in the art.

The terms “treatment” and “treating” as used herein cover any treatment of a condition or disease in an animal, preferably a mammal, more preferably a human, and includes: (i) inhibiting the microbial infection, eg arresting its proliferation; (ii) relieving the infection, eg causing a reduction in the severity of the infection; or (iii) relieving the conditions caused by the infection, eg symptoms of the infection. The terms “prevention” and preventing” as used herein cover the prevention or prophylaxis of a condition or disease in an animal, preferably a mammal, more preferably a human and includes preventing the microbial infection from occurring in a subject which may be predisposed to infection but has not yet been diagnosed as being infected.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The invention will now be described with reference to some specific examples and drawings. However, it is to be understood that the particularity of the following description is not to supercede the generality of the invention as hereinbefore described.

### EXAMPLES

### Physical Measurements

¹H NMR data were ¹H NMR spectra were recorded on a Varian Mercury 300 MHz spectrometer at room temperature in CD₂Cl₂ (>99.8%, Aldrich).

### Materials and Methods

Ethylene glycol, 1,10-phenanthroline (phen), potassium hexafluorophosphate (KPF₆), ammonium hexafluorophosphate (NH₄ PF₆), tetraethylammonium chloride were purchased from Aldrich and used as supplied. SP-Sephadex® C-25 cation-exchanger and Sephadex® LH-20 were obtained from Amersham Pharmacia Biotech. 3,4,7,8-Tetramethyl-1,10-phenanthroline (Me₄phen) was obtained from GFS chemicals.

### Synthesis of Ligands

Syntheses of Bridging Ligands “bbₙ”

The synthesis of ligands bbₙ (J. L. Morgan, C. B. Spillane, J. A. Smith, D. P. Buck, J. G. Collins and F. R. Keene, Dalton Trans. 2007, 4333-4342), Δ-[Ru(phen)₂(py)₂]{(−)-AsOtart}₂ (X. Hua and A. von Zelewsky, Inorg. Chem., 1995, 34, 5791-5797), [Ru(terpy)Cl₃](P. A. Adcock, F. R. Keene, R. S. Smythe, M. R. Snow, Inorg. Chem. 1984, 23, 2336-2343) and [Ru(Me₄-phen)₂Cl₂](T. Tagano, Inorg. Chim. Acta, 1992, 195, 221-225) were performed according to literature methods.

The ligands bb₂ (n=2), bb₅ (n=5), bb₇ (n=7), bb₁₀ (n=10), bb₁₂ (n=12), bb₁₄ (n=14) and bb₁₆ (n=16) are shown below:

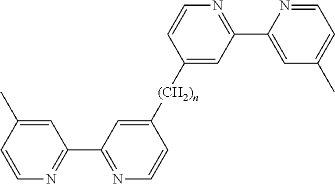

By way of example, to prepare mono-lithiated Me₂ bpy, Me₂ bpy (1.6 g, 8.7 mmol) was dissolved in dry THF (50 mL) under an inert atmosphere (Ar) at room temperature and the mixture cooled to −78° C. A solution of lithium diisopropylamide (9.6 mmol) in THF (10 mL) was added dropwise over the course of 30 min, and the mixture stirred for a further 1.5 h at −78° C., during which time the colour turned from white to dark brown-red. This mixture was brought to −10° C. over the course of 30 min.

For bb₁₂, 1,10-dibromodecane (1,12-dibromododecane for bb₁₄, 1,14-dibromotetradecane for bb₁₆; 4.3 mmol) was injected into the suspension of mono-lithiated Me₂ bpy. The reaction was brought to room temperature and left to stir under an inert atmosphere (Ar). A colour change from dark red to dark green to grey-green then to cream was typically observed within the first 2 h. After a further 24 h the reaction was quenched with water (10 mL), and the product extracted into diethyl ether (3×80 mL) and DCM (1×80 mL). The organic layers were combined, washed with water (1×50 mL), dried over anhydrous Na₂SO₄, filtered, and then evaporated to dryness in vacuo to yield a fluffy white powder in each case. The crude product was dissolved in a minimal volume of DCM and then loaded onto a silica gel column (230-400 mesh, 3 cm diam.×10 cm). The unreacted dibromopropane (pale yellow band) and Me₂ bpy (yellow band) were eluted using DCM. bb₁₂ and bb₁₄ (yellow band) and side products (yellow-brown band, suspected to be due to the di-lithiation of Me₂ bpy in the first step) were gradient-eluted using 1-10% (v/v) methanol in DCM. The purity and contents of each fraction were determined by TLC using 15% (v/v) methanol in DCM as a mobile phase. The purest fractions were combined and the solvent was removed in vacuo to yield fluffy white solids, bb₁₆, was recrystallised from boiling DCM after extraction. Yields: bb₁₂ (30%); bb₁₄ (33%); bb₁₆ (44%). Characterisation was achieved using ¹H NMR in CDCl₃. In some cases there was a presence of a small impurity of unreacted Me₂ bpy, which was difficult to completely eliminate by silica gel chromatography. The ligands were used for complex synthesis without further purification.

bb₁₂—¹H NMR (300 MHz, CDCl₃): d 8.55 (4H, dd, J=4.5, 3.0 Hz, bipy6); 8.24 (4H, s, bipy3); 7.14 (4H, dd, J=5.0, 1.0 Hz, bipy5); 2.70 (4H, t, 2×CH2bipy); 2.45 (6H, s, 2×CH3bipy); 1.28-1.70 (20H, m, 10×CH2).

bb₁₄—¹H NMR (300 MHz, CDCl₃): d 8.56 (4H, dd, J=4.5, 3.0 Hz, bipy6); 8.24 (4H, s, bipy3); 7.15 (4H, d, J=4.5, 1.0 Hz, bipy5); 2.70 (4H, t, 2×CH2bipy); 2.45 (6H, s, 2×CH3bipy); 1.26-1.72 (24H, m, 12×CH2).

bb₁₆—¹H NMR (300 MHz, CDCl₃): d 8.57 (4H, dd, J=4.5, 3.0 Hz, bipy6). 8.24 (4H, s, bipy3); 7.15 (4H, dd, J=5.0, 1.0 Hz, bipy5); 2.70 (4H, t, 2×CH₂bipy); 2.45 (6H, s, 2×CH₁₋₃bipy); 1.26-1.66 (28H, m, 14×CH₂).

Synthesis of Bridging Ligands bbNₙ

Analogous bridging ligands containing flexible polyamine chains were also synthesised. Using similar methodology to Sasaki and co-workers (I. Sasaki, M. Imberdis, A. Gaudemer, B. Drahi, D. Azhari and E. Amouyal, New J. Chem., 1994, 18, 759-764.) which provides polyamine-linked ligands via a condensation between 4-methyl-2,2′-bipyridine-4′-carboxaldehyde and appropriate primary amines, followed by the reduction of the resulting Schiff base products, in the present work 4-methyl-2,2′-bipyridine-4′-carboxaldehyde was reacted with 1,3-diaminopropane, diethylenetriamine (dien) and triethylenetetramine (trien) to afford polyamine-linked bis[4(4′-methyl-2,2′-bipyridine)]ligating groups (bbNₙ), as shown below:

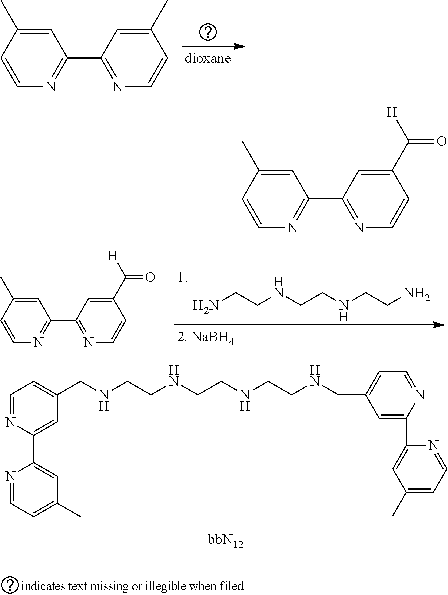 [SMILES: Cc1ccnc(-c2cc(C)ccn2)c1 ; Cc1ccnc(-c2cc(C=O)ccn2)c1 ; Cc1ccnc(-c2cc(C=O)ccn2)c1 ; NCCNCCNCCN ; Cc1ccnc(-c2cc(CNCCNCCNCCNCc3ccnc(-c4cc(C)ccn4)c3)ccn2)c1]

The ligands bbN₇ (n=7), bbN₉ (n=9) and bbN₁₂ (n=12) are shown below:

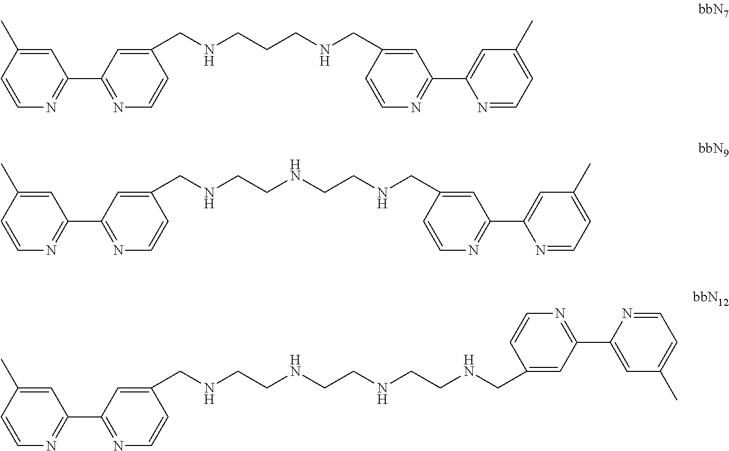 [SMILES: Cc1ccnc(-c2cc(CNCCCNCc3ccnc(-c4cc(C)ccn4)c3)ccn2)c1 ; Cc1ccnc(-c2cc(CNCCNCCNCc3ccnc(-c4cc(C)ccn4)c3)ccn2)c1 ; Cc1ccnc(-c2cc(CNCCNCCNCCNCc3ccnc(-c4cc(C)ccn4)c3)ccn2)c1]

A mixture of 4-methyl-2,2′-bipyridine-4′-carboxaldehyde (4.04 mmol) and the appropriate polyamine compound (1,3-diaminopropane, diethylenetriamine or triethylenetetramine, 2.02 mmol) was stirred in methanol (30 ml) at room temperature for 4 h. Sodium borohydride (4.04 mmol) was then added to the reaction with further stirring for 1 h. The solvent was removed and the crude residue redissolved in a minimum amount of water. The organic component was extracted three times with ethyl acetate, then washed with water and brine. After removing the solvent, the crude residue was chromatographed using silica gel with MeOH/ammonia (9:1) eluent to afford bbNₙ in 20-50% yield.

bbN₇—¹H NMR (300 MHz, CDCl₃): d 8.58 (2H, d, J=6.0, bipy6′); 8.51 (2H, d, J=5.0 Hz, bipy6); 8.21 (2H, s, bipy3); 8.31 (2H, s, bipy3′); 7.55 (2H, d, J=5.3, 1.0 Hz, bipy5′); 7.12 (2H, dd, J=5.0, 1.0 Hz, bipy5); 3.86 (4H, 2×CH₂bipy); 2.43 (6H, s, 2×CH₃bipy); 1.80 (2H, 2×—NH); 1.60, 2.61 (6H, m, 3×—CH₂).

bbN₉—¹H NMR (300 MHz, CDCl₃): d 8.58 (211, d, J=6.0, bipy6′); 8.51 (2H, d, J=5.0 Hz, bipy6); 8.31 (2H, s, bipy3′); 8.21 (2H, s, bipy3); 7.55 (2H, d, J=5.3, 1.0 Hz, bipy5′); 7.12 (2H, dd, J=5.0, 1.0 Hz, bipy5); 3.86 (4H, s, 2×CH₂bipy); 2.80 (8H, s, 4×—CH₂); 2.43 (6H, s, 2×CH₃bipy); 1.80 (3H, 3×—NH).

bbN₁₂—¹H NMR (300 MHz, CDCl₃): d 8.58 (2H, d, J=6.0, bipy6′); 8.51 (2H, d, J=5.0 Hz, bipy6); 8.31 (2H, s, bipy3′); 8.21 (2H, s, bipy3); 7.55 (2H, d, J=5.3, 1.0 Hz, bipy5′); 7.12 (2H, dd, J=5.0, 1.0 Hz, bipy5); 3.86 (4H, s, 2×CH₂bipy); 3.04 (4H, s, 4×—NH); 2.78 (12H, s, 6×—CH₂); 2.43 (6H, s, 2×CH₃bipy).

Synthesis of Ligands bbOₙ

Analogous bridging ligands containing (poly)ether groups (bbOₙ) were synthesised from the dibromopolyethoxy precursors (obtained from appropriate polyethylene glycols as reported in G. Bérubé, D. Rabouin, V. Perron, B. N'Zemba, R.-C. Gaudreault, S. Parent and E. Asselin, Steroids, 2006, 71, 911-921) as shown below:

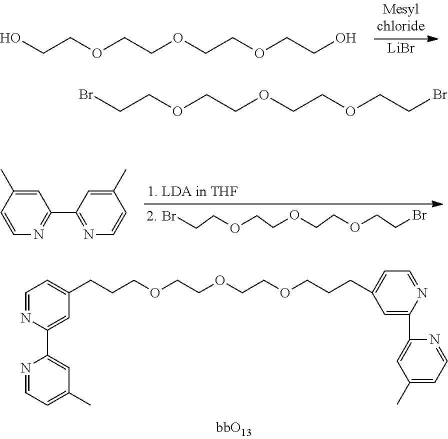 [SMILES: OCCOCCOCCOCCO ; "LiBr" ; BrCCOCCOCCOCCBr ; Cc1ccnc(-c2cc(C)ccn2)c1 ; BrCCOCCOCCOCCBr ; Cc1ccnc(-c2cc(CCCOCCOCCOCCCc3ccnc(-c4cc(C)ccn4)c3)ccn2)c1]

The ligands bbO₇ (n=7), bbO₁₀ (n=10), bbO₁₃ (n=13) and bbO₁₆ (n=16) are shown below:

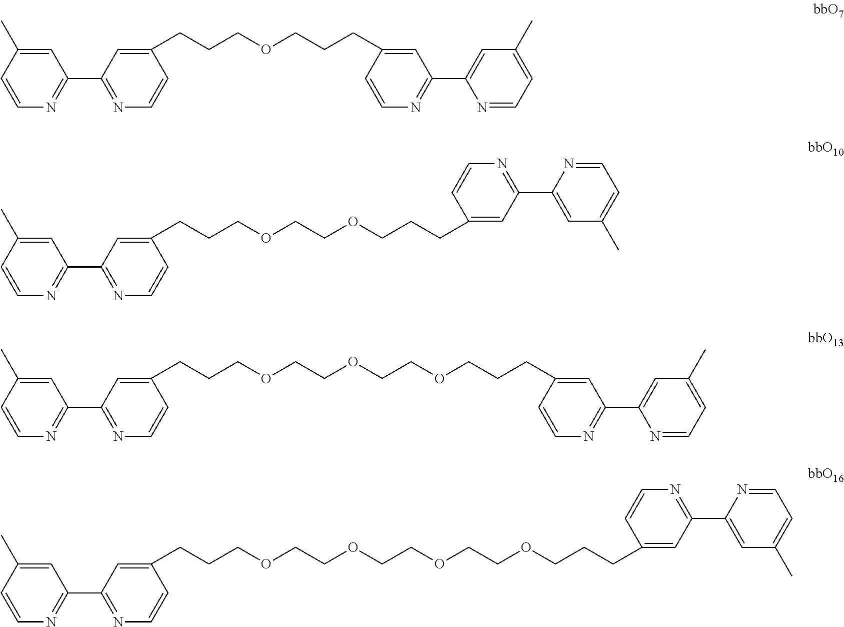 [SMILES: Cc1ccnc(-c2cc(CCCOCCCc3ccnc(-c4cc(C)ccn4)c3)ccn2)c1 ; Cc1ccnc(-c2cc(CCCOCCOCCCc3ccnc(-c4cc(C)ccn4)c3)ccn2)c1 ; Cc1ccnc(-c2cc(CCCOCCOCCOCCCc3ccnc(-c4cc(C)ccn4)c3)ccn2)c1 ; Cc1ccnc(-c2cc(CCCOCCOCCOCCOCCCc3ccnc(-c4cc(C)ccn4)c3)ccn2)c1]

Lithium diisopropylamide (2 M solution in heptane THF, 4.34 mmol) was added dropwise to a stirred solution of 4,4′-dimethyl-2,2′-bipyridine (8.68 mmol) in dry THF while maintaining the temperature at −78° C. (acetone/dry ice) under Ar. The dark-coloured mixture was further stirred for 2 h while the temperature was raised slowly to −10° C. An appropriate dibromopolyether compound (1,5-dibromo-3-oxapentane, 1,8-dibromo-3,6-dioxaoctane, 1,11-dibromo-3,6,9-trioxadecane or 1,14-dibromo-3,6,9,12-tetraoxatetradecane; 4.34 mmol) was then added and stirring continued for further 20 h. Water (30 ml) was added to the mixture, followed by extraction with diethyl ether and DCM. The organic phases were combined, washed with water and brine and dried over anhydrous Na₂SO₄ followed by filtration. Evaporation of the filtrate to dryness gave crude oil of bbOₙ, which was purified using a silica gel column with DCM-methanol (9:1) as the eluent. The pure bbOₙ was obtained in the middle band, while the first and the last were assigned as the 4,4′-dimethyl-2,2′-bipyridine and di-lithiation product impurities, respectively.

bbO₇—¹H NMR (300 MHz, CDCl₃): d 8.55 (4H, m, bipy6); 8.25 (4H, s, bipy3); 7.16 (4H, m, bipy5); 3.48 (4H, m, 2×CH₂O); 2.81 (4H, t, 2×CH₂bipy); 2.45 (6H, s, 2×CH₃bipy); 2.00 (4H, m, 2×CH₂).

bbO₁₀—¹H NMR (300 MHz, CDCl₃): d 8.55 (4H, m, bipy6); 8.25 (4H, s, bipy3); 7.16 (4H, m, bipy5); 3.51-3.62 (8H, m, 4×CH₂O); 2.81 (4H, t, CH₂bipy); 2.45 (6H, s, 2×CH₃bipy); 2.00 (4H, m, 2×CH₂).

bbO₁₃—¹H NMR (300 MHz, CDCl₃): d 8.55 (4H, m, bipy6); 8.25 (4H, s, bipy3); 7.16 (4H, m, bipy5); 3.51-3.62 (12H, m, 6×CH₂O); 2.81 (4H, t, CH₂bipy); 2.45 (6H, s, 2×CH₃bipy); 2.00 (4H, m, 2×CH₂).

bbO₁₆—¹H NMR (300 MHz, CDCl₃: d 8.55 (4H, m, bipy6); 8.25 (4H, s, bipy3); 7.16 (4H, m, bipy5); 3.51-3.65 (16H, m, 8×CH₂O); 2.81 (4H, t, CH₂bipy); 2.45 (6H, s, 2×CH₃bipy); 2.00 (4H, m, 2×CH₂).

### Synthesis of Complexes

Synthesis of Dinuclear Complexes ΔΔ- and ΛΛ-[{Ru(phen)₂}₂(μ-bbₙ)](PF₆)₄ (n=2, 5, 7, 10, 12, 14 and 16)

As used herein these complexes are also referred to by shorthand notation. For example ΛΛ-[{Ru(phen)₂}₂(μ-bb₁₂)] is referred to as ΔΔ-Rubb₁₂.

The syntheses of ΔΔ- and ΛΛ-[{Ru(phen)₂}₂(μ-bbₙ)]Cl₄ (n=12, 14, and 16) complexes were adapted from J. L. Morgan, C. B. Spillane, J. A. Smith, D. P. Buck, J. G. Collins and F. R. Keene, Dalton Trans., 2007, 4333-4342.

A typical procedure was as follows: Δ-[Ru(phen)₂(py)₂]{(−)-AsOtart}₂ (180 mg, 0.169 mmol) and bb₁₂ (43 mg, 0.085 mmol) were dissolved in ethylene glycol (4 mL) containing 10% (v/v) water, and stirred at 110° C. in the dark under an inert atmosphere (Ar) for 6 h. The completed reaction was cooled to room temperature, followed by the addition of water (10 mL) and ethanol (10 mL).

The dark red-orange solution was loaded onto an SP-Sephadex C-25 column (2 cm diam.×25 cm), and rinsed with water. Mononuclear impurities were eluted as a bright red-orange band using aqueous 0.3M NaCl solution, and the desired dinuclear species eluted as a bright red-orange band using 0.6 M NaCl solution. The dinuclear product was isolated from the eluate as its PF₆⁻ salt by a slow addition of saturated aqueous KPF₆, and then extraction into DCM.

The organic layer was washed with water, dried over anhydrous Na₂SO₄, and evaporated to dryness in vacuo to yield a bright red-orange precipitate, ΔΔ-[{Ru(phen)₂}₂(μ-bb₁₂)](PF₆)₄. The product was further purified by dissolving it in a minimal volume of acetone (AR), loading onto a silica gel column (1 cm diam.×5 cm), rinsed with acetone (AR) and then eluting with 5% (w/v) NH₄PF₆ in acetone (AR). An equal volume of water was added to the red-orange eluate and the acetone removed in vacuo. The resulting bright orange precipitate was collected by filtration and washed with cold water (˜50 mL).

The precipitate was then converted into its chloride salt by stirring it in an aqueous solution using AmberliteR® IRA-400 (chloride form) anion-exchange resin. The resin was removed by filtration, and the dark red filtrate was freeze dried to obtain a fluffy bright orange powder. Typical yield ˜20%. The corresponding AA complexes were synthesised as described above, by using Λ-[Ru(phen)₂(py)₂]]{(+)-AsOtart}₂ as the mononuclear precursor. In the cases of [{Ru(phen)₂}₂(μ-bb₁₄)]Cl₄ and [{Ru(phen)₂}₂(μ-bb₁₆)]Cl₄, the eluents were required to include 10% acetone to keep the complexes solubilised during SP-Sephadex C-25 purification.

Circular dichroism spectra and 1D ¹H NMR were consistent with those reported in J. L. Morgan, C. B. Spillane, J. A. Smith, D. P. Buck, J. G. Collins and F. R. Keene, Dalton Trans., 2007, 4333-4342 for bb₂, bb₅, bb₇, and bb₁₀ bridged species.

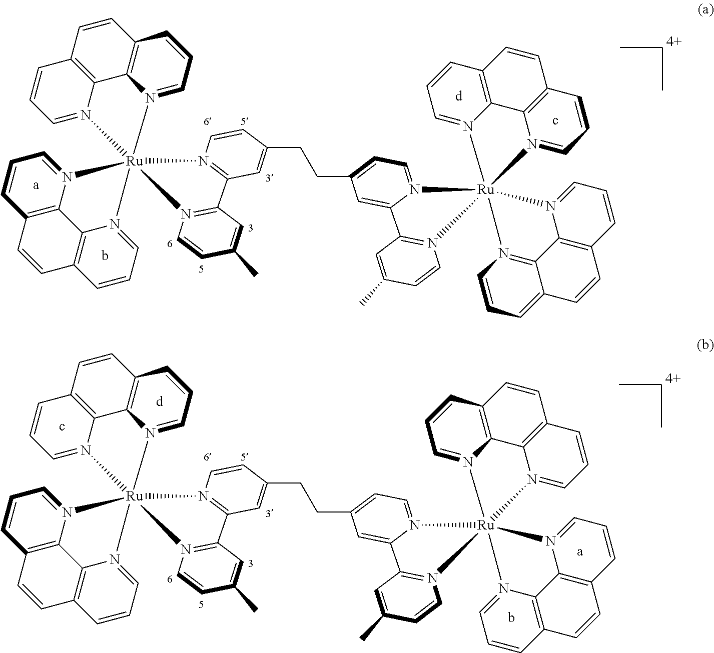

Diastereoisomeric forms of [{Ru(phen)₂}₂(μ-bb₂)]⁴⁺: (a) rac {ΔΔ(≡ΛΛ)}; (b) meso. Hydrogen atoms are omitted for clarity; the notation shown is used in the assignment of the ¹H NMR spectra in D₂O.

The hexafluorophosphate salts were able to be metathesised to the water-soluble bromide salts by dissolution in a minimum volume of acetone and the addition of [(n-C₄H₉)₄N]Br until complete precipitation had occurred. The products were filtered and washed with cold acetone. In some cases the hexafluorophosphate salts were converted to the water-soluble chloride salts by stirring with Dowex ion-exchange resin in water. After filtering, the water solution was freeze-dried to obtain a fluffy orange powder.

[{Ru(phen)₂}₂(μ-bb₂)](PF₆)₄.3H₂O. Found: C, 45.0; H, 3.41; N, 8.3%. Calc. for C₇₂H₅₄N₁₂Ru₂P₄F₂₄.3H₂O: C, 45.0; H, 3.14; N, 8.7%. UV/Vis (MeCN)—λₘₐₓ/nm (ε/M⁻¹cm⁻¹): 451 (0.28×105) 285 sh (0.71×105), 264 (1.37×105).

Meso-[{Ru(phen)₂}₂(μ-bb₂)](PF₆)₄: 1H NMR (300 MHz, CD₃CN): d 8.65 (4H, dd, J=8.1, 1.0 Hz, H7); 8.54 (4H, dd, J=8.1, 1.0 Hz, H4); 8.51 (2H, dd, J=1.2, −0.5 Hz, bpy3); 8.45 (2H, dd, J=1.2, −0.5 Hz, bpy3′); 8.28-8.21 (8H, m, H5,6); 8.20 (4H, dd, J=5.0, 1.0 Hz, H9); 7.89 (4H, dd, J=5.0, 1.0 Hz, H2); 7.76 (4H, ddd, J=8.1, 5.0, 1.0 Hz, H8); 7.59-7.55 (6H, m, H3, bpy6); 7.50 (2H, d, J=5.7 Hz, bpy6′); 7.21 (2H, dd, J=5.7, 1.0 Hz, bpy5); 7.14 (2H, dd, J=5.7, 1.0 Hz, bpy5), 3,13, (4H, s, 2×CH₂), 2.52 (6H, s, 2×Me).

Rac-[{Ru(phen)₂}₂(μ-bb₂)](PF₆)₄: ¹H NMR (300 MHz, CD₃CN): d 8.66 (4H, dd, J=8.1, 1.0 Hz, H7); 8.57 (4H, dd, J=8.1, 1.0 Hz, H4); 8.53 (2H, dd, J=1.2, −0.5 Hz, bpy3); 8.46 (2H, dd, J=1.2, ˜0.5 Hz, bpy3′); 8.29-8.21 (8H, m, H5,6); 8.21 (4H, dd, J=5.0, 1.0 Hz, H9); 7.89 (4H, dd, J=5.0, 1.0 Hz, H2); 7.77 (4H, ddd, J=8.1, 5.0, 1.0 Hz, H8); 7.59-7.55 (6H, m, H3, bpy6); 7.50 (2H, d, J=5.7 Hz, bpy6′); 7.21 (2H, dd, J=5.7, 1.0 Hz, bpy5); 7.14 (2H, dd, J=5.7, 1.0 Hz, bpy5), 3,14, (4H, s, 2×CH2), 2.52 (6H, s, 2×Me). CD {λ/nm (Δe/cm⁻¹M⁻¹) CH₃CN}—ΔΔ: 468 (−24), 420 (19), 283 (−267), 269 (−296), 258 (367). ΛΛ: 468 (23), 420 (−25), 283 (246), 269 (281), 258 (−354). [{Ru(phen)₂}₂(μ-bb₅)](PF₆)₄.7H₂O. Found: C, 44.1; H, 3.43; N, 8.2%. Calc. for C₇₅H₆₀N₁₂Ru₂P₄F₂₄.7H₂O: C, 44.2; H, 3.66; N, 8.2%.

Meso-[{Ru(phen)₂}₂(μ-bb₅)](PF₆)₄: ¹H NMR (300 MHz, CD3CN): d 8.66 (4H, dd, J=8.1, 1.0 Hz, H7); 8.54 (4H, dd, J=8.1, 1.0 Hz, H4); 8.42 (2H, dd, J=1.2, −0.5 Hz, bpy3); 8.39 (2H, dd, J=1.2, ˜0.5 Hz, bpy3); 8.28-8.21 (8H, m, H5,6); 8.22 (4H, dd, J=5.0, 1.0 Hz, H9); 7.89 (4H, dd, J=5.0, 1.0 Hz, H3); 7.79 (4H, ddd, J=8.1, 5.1, 1.0 Hz, H8); 7.57-7.47 (8H, m, H3, bpy6); 7.15-7.11 (4H, m, bpy5); 2.79, (4H, bt, J=5.1 Hz, 2×CH2-bpy); 2.52 (6H, s, 2×Me); 1.77-1.65 (6H, m, J=Hz, 3×CH2).

Rac-[{Ru(phen)₂}₂(μ-bb₅)](PF₆)₄: ¹H NMR (300 MHz, CD3CN): d 8.65 (4H, dd, J=8.1, 1.0 Hz, H7); 8.55 (4H, dd, J=8.1, 1.0 Hz, H4); 8.42 (2H, dd, J=1.2, −0.5 Hz, bpy3); 8.38 (2H, dd, J=1.2, ˜0.5 Hz, bpy3); 8.28-8.22 (8H, m, H5,6); 8.21 (4H, dd, J=5.0, 1.0 Hz, H9); 7.89 (4H, dd, J=4.5, 1.0 Hz, H3); 7.80 (4H, ddd, J=8.1, 5.1, 1.0 Hz, H8); 7.56 (4H, dd, J=8.2, 5.1 H3); 7.50 (4H, dd, J=5.4, 5.1, bpy6) 7.18-7.08 (4H, m, bpy5); 2.79, (4H, bt, J=˜7 Hz, 2×CH2-bpy); 2.53 (6H, 2×Me); 1.78-1.66 (6H, m, J=Hz, 3×CH2). CD {k/nm (De/cm-1M-1) CH3CN}—ΔΔ: 466 (−24), 418 (22), 285 (−233), 268 (−312), 260 (334). ΛΛ: 466 (26), 418 (−20), 285 (239), 268 (318), 258 (−345).

[{(Ru(phen)₂}₂(μ-bb₇)](PF₆)₄.2(acetone).6H₂O: Found C, 45.8; H. 3.83; N, 7.5%. Calc. for C₇₇H₆₄N₁₂Ru₂P₄F₂₄.2C₃H₆O.6H₂O: C, 46.1; H, 4.10; N, 7.8%.

Rac-[{Ru(phen)₂}₂(μ-bb₇)](PF₆)₄: ¹H NMR (300 MHz, CD₃CN): d 8.64 (4H, ddd, J=8.1, 4.8, 1.0 Hz, H8); 8.54 (2H, dd, J=8.1, 1.0 Hz, H4); 8.42 (2H, dd, J=1.2, ˜0.5 Hz, bpy3); 8.37 (2H, dd, J=1.2, ˜0.5 Hz, bpy3); 8.28-8.22 (8H, m, H5,6); 8.21 (4H, dd, J=5.0, 1.0 Hz, H7); 7.89 (4H, dd, J=4.5, 1.0 Hz, H2); 7.80 (4H, ddd, J=8.1, 5.1, 1.0 Hz, H9); 7.57 (4H, dd, J=8.2, 5.1 H3); 7.50 (4H, dd, J=5.4, 5.1, bpy6); 7.16-7.08 (4H, m, bpy5); 2.77, (4H, bt, J=˜7 Hz, 2×CH2-bpy); 2.53 (6H, s, 2×Me); 1.74-1.58 (6H, m, J=Hz, 3×CH2). CD {λ/nm (Δe/cm⁻¹M⁻¹) CH₃CN}—ΔΔ: 465 (−23), 417 (20), 284 (−221), 268 (−296), 260 (313). ΛΛ: 465 (24), 417 (20), 284 (231), 268 (293), 260 (−336).

[{Ru(phen)₂}₂(μ-bb₁₀)](PF₆)₄.10H₂O. Found C, 44.5; H, 3.96; N, 7.4%. Calc. for C₈₀H₇₀N₁₂Ru₂P₄F₂₄.10H₂O: C, 44.3; H, 4.20; N, 7.8%.

Meso-[{Ru(phen)₂}₂(μ-bb₁₀)](PF₆)₄: ¹H NMR (300 MHz, CD₃CN): d 8.66 (4H, dd, J=8.1, 1.0 Hz, H7); 8.55 (4H, dd, J=8.1, 1.0 Hz, H4); 8.42 (2H, dd, J=1.2, ˜0.5 Hz, bpy3); 8.37 (2H, dd, J=1.2, ˜0.5 Hz, bpy3); 8.28-8.24 (8H, m, H5,6); 8.21 (4H, dd, J=5.0, 1.0 Hz, H9); 7.89 (4H, ddd, J=5.0, 1.0, ˜0.3 Hz, H2); 7.79 (4H, ddd, J=8.1, 5.1, 1.0 Hz, H8); 7.56 (4H, ddd, J=8.1, 5.7, ˜0.3 Hz, H3); 7.50 (4H, dd, J=5.3, 3.6 Hz, bpy6); 7.15-7.08 (4H, m, bpy5); 2.77, (4H, bt, J=5.1 Hz, 2×CH2-bpy); 2.53 (6H, s, 2×Me); 1.73-1.60 (6H, m, J=Hz, 2×CH2); 1.40-1.24 (12H, m, 6×CH2).

Rac-[{Ru(phen)₂}₂(μ-bb₁₀)](PF₆)₄: ¹H NMR (300 MHz, CD₃CN): d 8.66 (4H, dd, J=8.1, 1.0 Hz, H7); 8.55 (4H, dd, J=8.1, 1.0 Hz, H4); 8.42 (2H, dd, J=1.2, ˜0.5 Hz, bpy3); 8.37 (2H, dd, J=1.2, ˜0.5 Hz, bpy3); 8.28-8.24 (8H, m, H5,6); 8.21 (4H, dd, J=5.0, 1.0 Hz, H9); 7.89 (4H, ddd, J=5.0, 1.0, ˜0.3 Hz, H2); 7.79 (4H, ddd, J=8.1, 5.1, 1.0 Hz, H8); 7.56 (4H, ddd, J=8.1, 5.7, ˜0.3 Hz, H3); 7.50 (4H, dd, J=5.3, 3.6 Hz, bpy6); 7.15-7.08 (4H, m, bpy5); 2.77, (4H, bt, J=5.1 Hz, 2×CH2-bpy); 2.53 (6H, s, 2×Me); 1.73-1.60 (6H, m, J=Hz, 2×CH2); 1.40-1.24 (12H, m, 6×CH2). CD {k/nm (De/cm-1M-1) CH₃CN}—ΔΔ: 465 (−21), 418 (17), 284 (−203), 268 (−254), 259 (289). AA: 465 (22), 418 (−18), 284 (202), 268 (251), 259 (−290).

ΔΔ-[{Ru(phen)₂}₂(μ-bb₁₂)](PF₆)₄. H₂O. Anal. Found C, 48.8; H, 3.82: N, 8.5%. Calcd. for C₈₂H₇₆N₁₂F₂₄OP₄Ru₂: C, 48.6; H, 3.78; N, 8.3%. ¹H NMR (300 MHz, CD₃CN): d 8.69 (4H, d, J=8.5 Hz, H2, H9); 8.58 (4H, d, J=9.0 Hz, H2, H9); 8.42 (4H, d, J=14.0 Hz, bipy6); 8.27 (8H, dd, J=3.0, 1.0, H4, H7); 8.24 (4H, m, bipy3); 7.92 (4H, m, H5, H6); 7.82 (4H, m, H5, H6); 7.58 (4H, dd, J=8.0, 5.3 Hz, H3, H8); 7.52 (4H, dd, J=5.8, 4.0 Hz, H3, H8); 7.15 (4H, m, bipy5); 2.80 (4H, t, 2×CH₂bipy); 2.55 (6H, s, CH₃bipy); 1.29-1.69 (20H, m, 10×CH₂). CD {λ/nm(Δε/cm⁻¹M⁻¹), Cl⁻ salt in H₂O}: ΔΔ: 473.5 (−28.6); 419 (28.5); 287.5 (−277); 269 (−227); 259.5 (292); 218 (52.9). ΛΛ: 471 (29.0); 415.5 (−26.3); 287.5 (282); 268.5 (242); 260 (−315); 218.5 (−57.0).

ΔΔ-[{Ru(phen)₂}₂(μ-bb₁₄)](PF₆)₄.H₂O. Anal. Found C, 48.9; H, 3.75: N, 8.6%. Calcd. for C₈₄H₈₀N₁₂F₂₄OP₄Ru₂: C, 49.1; H, 3.92; N, 8.2%. ¹H NMR (300 MHz, CD₃CN): d 8.68 (4H, d, J=8.3 Hz, H2, H9); 8.58 (4H, d, J=8.2 Hz, H2, H9); 8.42 (4H, d, J=14.0 Hz, bipy6); 8.28 (8H, dd, J=3.5, 1.0, H4, H7); 8.24 (4H, m, bipy3); 7.91 (4H, m, H5, H6); 7.83 (4H, m, H5, H6); 7.59 (4H, dd, J=8.5, 5.0 Hz, H3, H8); 7.52 (4H, dd, J=6.0, 4.0 Hz, H3, H8); 7.15 (4H, m, bipy5); 2.80 (4H, t, 2×CH₂bipy); 2.55 (6H, s, CH₃bipy); 1.29-1.69 (24H, m, 12×CH₂). CD {λ/nm(Δε/cm⁻¹M⁻¹), Cl⁻ salt in H₂O)}: ΔΔ: 472.5 (−31.9); 417.5 (24.2); 286 (−279); 269 (−296); 260(353); 216 (62.1). ΛΛ: 468.5 (32.5); 415 (−22.8); 286(276); 269(293); 260 (−352); 216.5 (−63.5.

ΔΔ-[{Ru(phen)₂}₂(μ-bb₁₆)](PF₆)₄.3H₂O.NH₄ PF₆. Anal. Found C, 44.5; H, 3.38: N, 7.8%. Calcd. for C₈₆H₉₂N₁₃F₃₀O₃P₅Ru₂: C, 45.2; H, 4.07; N, 8.0%. ¹H NMR (300 MHz, CD₃CN): d 8.71 (4H, d, J=8.3 Hz, H2, H9); 8.59 (4H, d, J=8.2 Hz, H2, H9); 8.52 (4H, d, J=14.0 Hz, bipy6); 8.29 (8H, d, J=3.0 Hz, H4, H7); 8.24 (4H, m, bipy3); 7.92 (4H, m, H5, H6); 7.84 (4H, m, H5, H6); 7.60 (4H, dd, J=8.5, 5.0 Hz, H3, H8); 7.52 (4H, t, H3, H8); 7.15 (4H, m, bipy5); 2.80 (4H, t, 2×CH₂bipy); 2.55 (6H, s, CH₃bipy); 1.70-1.27 (28H, m, 14×CH₂). CD {λ/nm(Δε/cm⁻¹M⁻¹), Cl⁻ salt in H₂O}: ΔΔ: 468.5 (−35.6); 418 (22.7); 285.5 (−292); 270 (−306); 260(357); 218.5 (72.1). AA: 468 (36.0); 417.5 (−20.5); 285.5 (285); 269.5 (306); 260 (−343); 219.5 (−70.4).

Representative synthesis of [{Ru(Me₄phen)₂}₂(Δ-bbₙ)]Cl₄ (n=7, 12, 16)

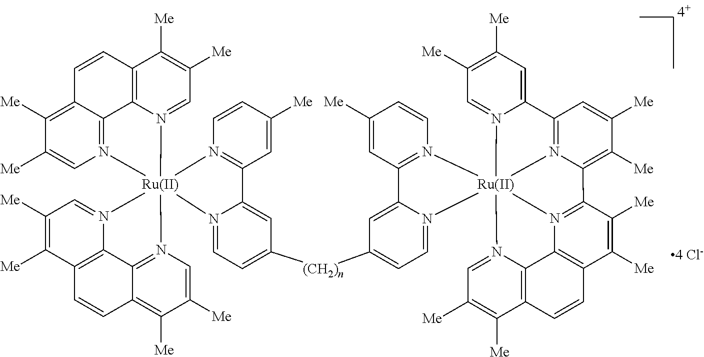

[Ru(Me₄phen)₂Cl₂] (0.15 mmol) and bbₙ (0.075 mmol) were refluxed in EtOH/water (1:1, 20 ml) for four hours. After cooling, the solvent was evaporated under reduced pressure until half of the original volume. The mixture was then loaded onto a Sephadex C-25 cation exchange column, eluted with water, 0.3 M NaCl then with 1.0 M NaCl to remove the impurities. Elution with 1.0 M NaCl containing 5% acetone gave the pure [{Ru(Me₄phen)₂}₂(μ-bb₇)]²⁺. For [{Ru(Me₄phen)₂}₂(μ-bb₁₂)]²⁺, the complex was obtained by elution with 1.0 M NaCl containing 10% acetone. For [{Ru(Me₄Phen)₂}₂(μ-bb₁₆)]²⁺, the complex was obtained by elution with 1.0 M NaCl containing 20% acetone. After acetone removal, excess KPF₆ was added causing the precipitation of the PF₆⁻ salt of the complex which was then extracted into dichloromethane followed by evaporation to dryness to give the corresponding [{Ru(Me₄phen)₂}₂(μ-bbₙ)](PF₆)₂. The PF₆⁻ salt was converted to the chloride by dissolving the solid in the minimum amount of acetone followed by drop wise addition of the saturated solution of tetraethylammonium chloride in acetone while stirring for half an hour. The resulting solid was filtered and washed with acetone and dried under reduced pressure to afford [{Ru(Me₄phen)₂}₂(μ-bbₙ)](Cl)₄. Typical yield: 30-50%.

[{Ru(Me₄phen)₂}₂(Δ-bb₇)](PF₆)₂ ¹H NMR (300 MHz, CD₂Cl₂) δ 8.30 (m); 8.90 (s), 8.85 (s); 8.55 (d), 8.51 (t); 7.20 (m); 2.90-2.85 (m), 2.51 (s), 2.49 (d), 2.30 (s), 1.80 (s), 1.33 (s).

Synthesis of Dinuclear Complexes ΔΔ- and ΛΛ-[{Ru(phen)₂}₂(μ-bbXₙ)]b⁺ (X═N, O)

Using similar synthetic techniques to those described above to produce the alkylene bridged complexes stereoselectively, dinuclear complexes ΔΔ-[{Ru(phen)₂}₂(μ-bbXₙ)]⁴⁺ (X═N, O) were produced. These complexes were separated from mononuclear species and other impurities using an SP-Sephadex C-25 cation exchange column with a gradient concentration of aqueous sodium chloride solution as eluent. In contrast to the chromatographic purification of ΔΔ-[{Ru(phen)₂}₂(μ-bbₙ)]⁴⁺ and ΔΔ-[{Ru(phen)₂}₂(μ-bbOₙ)]⁴⁺, difficulties were encountered with ΔΔ-[{Ru(phen)₂}₂(μ-bbNₙ)]⁴⁺ species as there was a significant broadening of the bands, presumably due to the protonation of the free amine moieties. Compared with the alkylene- and (poly)ether-bridged analogues, the purification of ΔΔ-[{Ru(phen)₂}₂(μ-bbNₙ)]⁴⁺ species generally required much slower flow rates and higher concentration of electrolyte in the eluent. The protonation of the free secondary amine groups during the separation processes is not unexpected at the neutral pH values of these procedures, and was exemplified by the microanalytical data of the isolated dinuclear species which were consistent with bbN₁₂ being protonated to give the ΔΔ-[{Ru(phen)₂}₂(μ-bbH₄N₁₂)]⁸⁺ form rather than non-protonated ΔΔ-[{Ru(phen)₂}₂(μ-bbN₁₂)]⁴⁺:

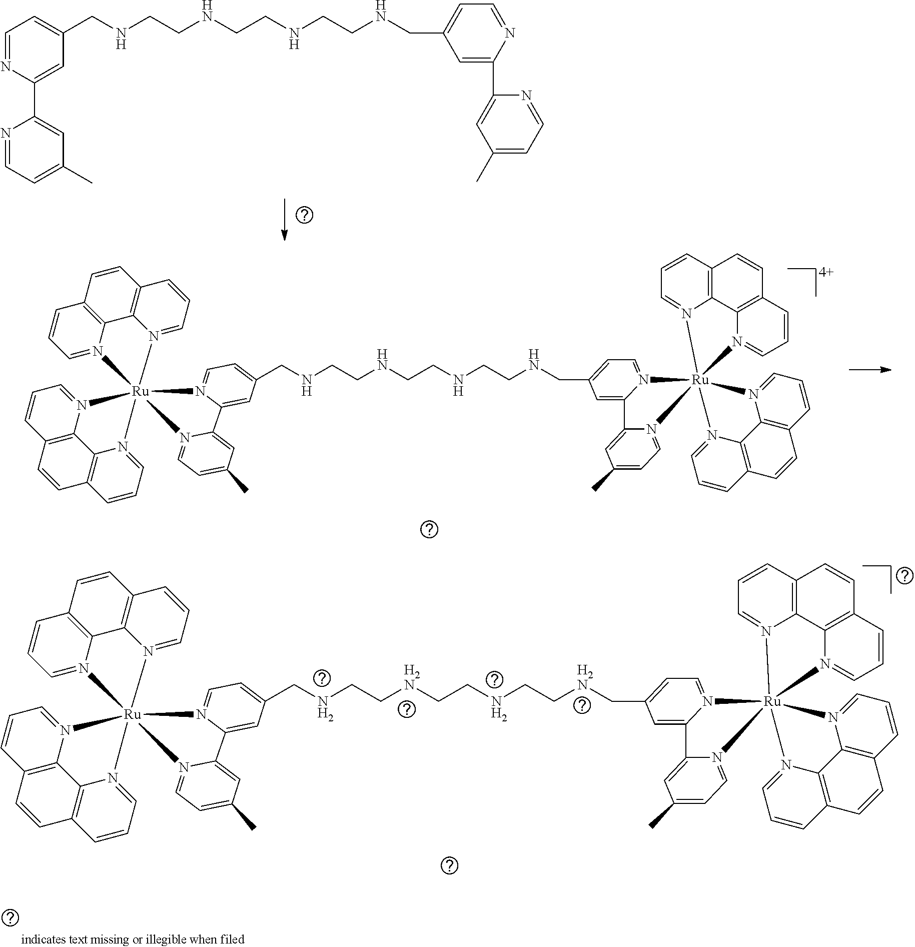

### Synthesis of Dinuclear Complexes ΔΔ-[{Ru(phen)₂}₂(μ-bbXₙ)](PF₆)₄ (RubbXₙ; X═N, O)

As used herein these complexes are also referred to by shorthand notation. For example ΔΔ-[{Ru(phen)₂}₂(μ-bbN₇)] is referred to as ΔΔ-RubbN₇.

Δ-[Ru(phen)₂(py)₂]{(−)−AsOtart}₂ (0.18 g, 0.169 mmol) and bbXₙ (0.085 mmol) were dissolved in ethylene glycol (4 mL) containing 10% (v/v) water, and stirred at 110° C. in the dark under Ar for 6 h. The completed reaction was cooled to room temperature, followed by the addition of water (10 mL). The dark red-orange solution was loaded onto a SP-Sephadex C-25 column and washed with water. Elution with 0.3 M NaCl solution removed the mononuclear impurities. The desired dinuclear species were eluted with 0.6 M NaCl solution, except for the one case of ΔΔ-[{Ru(phen)₂}₂(μ-bbN₁₂)]Cl₄, which was obtained by using 1.0 M NaCl solution. Solid KPF₆ was added to the eluates, followed by extraction into DCM. The organic layer was washed with water, dried over anhydrous Na₂SO₄, and evaporated to dryness in vacuo to yield a bright red-orange precipitate, ΔΔ-[{Ru(phen)₂}₂(μ-bbXₙ)](PF₆)₄. The complexes were further purified by dissolving them in minimum amount of acetone, loading onto Sephadex LH-20 and then eluting with acetone.

The solvent was removed to dryness and the resultant bright orange complexes then converted to their chloride salts by stirring them to in aqueous solution using Amberlite® IRA-400 (chloride form) anion-exchange resin. The resin was removed by filtration, and the filtrate was freeze-dried to afford a fluffy bright orange ΔΔ-[{Ru(phen)₂}₂(μ-bbXₙ)]Cl₄ in 20-25% yield. ¹H NMR (aromatic regions) and CD spectral data of these complexes are consistent with previously reported ΔΔ-[{Ru(phen)₂}₂(μ-bbₙ)]complexes.

ΔΔ-[{Ru(phen)₂}₂(μ-bbH₂N₇)](PF₆)₆.4H₂O-acetone. Anal. Found C, 39.8; H, 3.65: N, 8.5%. Calcd. for C₇₈H₇₈N₁₄F₃₆O₅P₆Ru₂: C, 39.6; H, 3.33; N, 8.3%. ¹H NMR (300 MHz, CD₃CN): d 8.73-8.55 (12H, m, H2, H9, bipy6); 8.28 (8H, d, J=3.0 Hz, H4, H7); 8.24 (4H, m, bipy3); 7.92 (4H, m, H5, H6) 7.69-7.53 (8H, m, H3, H8); 7.30 (2H, d, J=4.3 Hz, bipy5′); 7.18 (2H, d, J=6.0 Hz, bipy5); 4.14 (4H, s, 2×CH₂bipy); 3.02 (4H, s, 2×CH₂N); 2.55 (6H, s, 2×CH₃bipy); 1.88 (2H, m, CH₂). CD {λ/nm (Δε/cm⁻¹M⁻¹), Cl⁻ salt in H₂O} 468 (−23), 420 (21), 286.5 (−188), 268.5 (−246), 259.5 (294), 216.5 (58).

ΔΔ-[{Ru(phen)₂}₂(μ-bbH₄N₁₂)](PF₆)₈.2H₂O. Anal. Found C, 35.8; H, 2.85: N, 7.8%. Calcd. for C₇₈H₇₈N₁₆F₄₈O₂P₈Ru₂: C, 35.6; H, 2.99; N, 8.5%. ¹H NMR (300 MHz, CD₃CN): d 8.70-8.56 (12H, m, H2, H9, bipy6); 8.28 (8H, d, J=3.0, H4, H7); 7.92 (4H, m, H5, H6); 7.83 (4H, m, H5, H6); 7.65-7.52 (8H, m, H3, H8); 7.31 (2H, d, J=5.0 Hz, bipy6′); 7.15 (2H, d, J=5.0 Hz, bipy6); 4.07 (4H, s, 2×CH₂bipy). 3.10 (12H, s, 6×CH₂N); 2.53 (6H, s, 2×CH₃bipy). CD {λ/nm (Δε/cm⁻¹M⁻¹), Cl⁻ salt in H₂O} 467.5 (−23), 417.5 (21), 288 (−194), 268.5 (−253), 259.5 (292), 219.5 (55).

ΔΔ-[{Ru(phen)₂}₂(μ-bbO₇)](PF₆)₄.2H₂O. Anal. Found C, 46.1; H, 3.11: N, 8.1%. Calc. for C₇₆H₆₆N₁₂F₂₄O₃P₄Ru₂: C, 46.2; H, 3.36; N, 8.5%. ¹H NMR (300 MHz, CD₃CN): d 8.68 (4H, dd, J=8.0, 1.0 Hz, H2, H9); 8.58 (4H, dt, J=9.0 Hz, H2, H9); 8.43 (4H, d, J=8.0 Hz, bipy6); 8.28 (8H, m, H4, H7); 8.23 (4H, m, bipy3); 7.92 (4H, m, H5, H6); 7.83 (4H, m, H5, H6); 7.61-7.51 (8H, m, H3, H8); 7.15 (4H, d, J=6.0 Hz, bipy5); 3.48 (4H, t, 2×CH₂O); 2.86 (4H, t, 2×CH₂bipy); 2.53 (6H, s, 2×CH₃bipy); 1.96 (4H, m, 2×CH₂). CD {λ/nm (Δε/cm⁻¹M⁻¹), Cl⁻ salt in H2O} 469.5 (−20), 420 (19), 288 (−212), 268.5 (−211), 259.5 (262), 216.5 (53).

ΔΔ-[{Ru(phen)₂}₂(μ-bbO₁₀)](PF₆)₄.H₂O. Anal. Found C, 46.7; H, 3.22: N, 8.0%. Calcd., for C₇₈H₆₈N₁₂F₂₄O₃P₄Ru₂: C, 46.8; H, 3.42; N, 8.4%. ¹H NMR (300 MHz, CD₃CN): d 8.70 (4H, d, J=7.0 Hz, H2, H9); 8.58 (4H, d, J=9.0 Hz, H2, H9); 8.44 (4H, d, J=10.3 Hz, bipy6); 8.29 (8H, m, H4, H7); 8.24 (4H, m, bipy3); 7.92 (4H, m, H5, H6); 7.83 (4H, m, H5, H6); 7.61-7.51 (8H, m, H3, H8); 7.15 (4H, d, J=5.0 Hz, bipy5); 3.53-3.50 (8H, m, 4×CH₂O); 2.87 (4H, t, CH₂bipy); 2.55 (6H, s, 2×CH₃bipy); 1.97 (4H, m, 2×CH₂). CD {λ/nm (Δε/cm⁻¹M⁻¹), Cl⁻ salt in H₂O} 468.5 (−29), 416 (27), 286.5 (−286), 268.5 (−297), 259.5 (300), 217 (71).

Δ-[{Ru(phen)₂}₂(μ-bbO₁₃)](PF₆)₄.2H₂O. Anal. Found C, 46.7; H, 3.51: N, 7.8%. Calcd. for C₈₀H₇₄N₁₂F₂₄O₅P₄Ru₂: C, 46.5; H, 3.61; N, 8.1% ¹H NMR (300 MHz, CD₃CN): d 8.61 (4H, d, J=8.0 Hz, H2, H9); 8.58 (4H, d, J=8.0 Hz, H2, H9); 8.44 (4H, d, J=10.3 Hz, bipy6); 8.29 (8H, m, H4, H7); 8.24 (4H, m, bipy3); 7.92 (4H, m, H5, H6); 7.83 (4H, m, H5, H6); 7.61-7.51 (8H, m, H3, H8); 7.15 (4H, d, J=5.0 Hz, bipy5); 3.53-3.48 (12H, s, 6×CH₂O); 2.85 (4H, t, CH₂bipy); 2.54 (6H, s, 2×CH₃bipy); 1.96 (4H, m, 2×CH₂); CD {λ/nm (Δε/cm⁻¹M⁻¹), Cl⁻ salt in H₂O} 469 (−26), 420 (23), 286.5 (−254), 269 (−276), 259.5 (300), 217 (67).

ΔΔ-[{Ru(phen)₂}₂(μ-bbO₁₆)](PF₆)₄H₂O. Anal. Found C, 47.2; H, 3.48: N, 7.6%. Calcd. for C₈₂H₇₆N₁₂F₂₄O₅P₄Ru₂: C, 47.1; H, 3.66; N, 8.0% ¹H NMR (300 MHz, CD₃CN): d 8.68 (4H, d, J=8.0 Hz, H2, H9); 8.58 (4H, d, J=8.0 Hz, H2, H9); 8.44 (4H, d, J=10.3 Hz, bipy6); 8.29 (8H, m, H4, H7); 8.24 (4H, m, bipy3); 7.92 (4H, m, H5, H6); 7.83 (4H, m, H5, H6); 7.61-7.51 (8H, m, H3, H8); 7.15 (4H, d, J=5.0 Hz, bipy5); 3.53-3.48 (16H, m, 8×CH₂O); 2.87 (4H, t, CH₂bipy); 2.55 (6H, s, 2×CH₃bipy); 1.98 (4H, m, 2×CH₂); CD {λ/nm (Δε/cm⁻¹M⁻¹), Cl⁻ salt in H₂O} 470.5 (−27), 415.5 (25), 28.5 (−273), 269 (−294), 260.5 (300), 216.5 (67).

The synthesis of the above dinuclear (poly)oxy and polyamino complexes generally produced mononuclear species as side products. In the cation exchange chromatography purification procedure, the mononuclear Δ-[Ru(phen)₂(bbN₇)]²⁺ species was well separated from the dinuclear analogue ΔΔ-[{Ru(phen)₂}₂(μ-bbH₂N₇)]⁶⁺, although the microanalysis of the isolated mononuclear product suggested that it also underwent protonation to form a higher charged complex Δ-[Ru(phen)₂(bbH₄N₇)]⁶⁺:

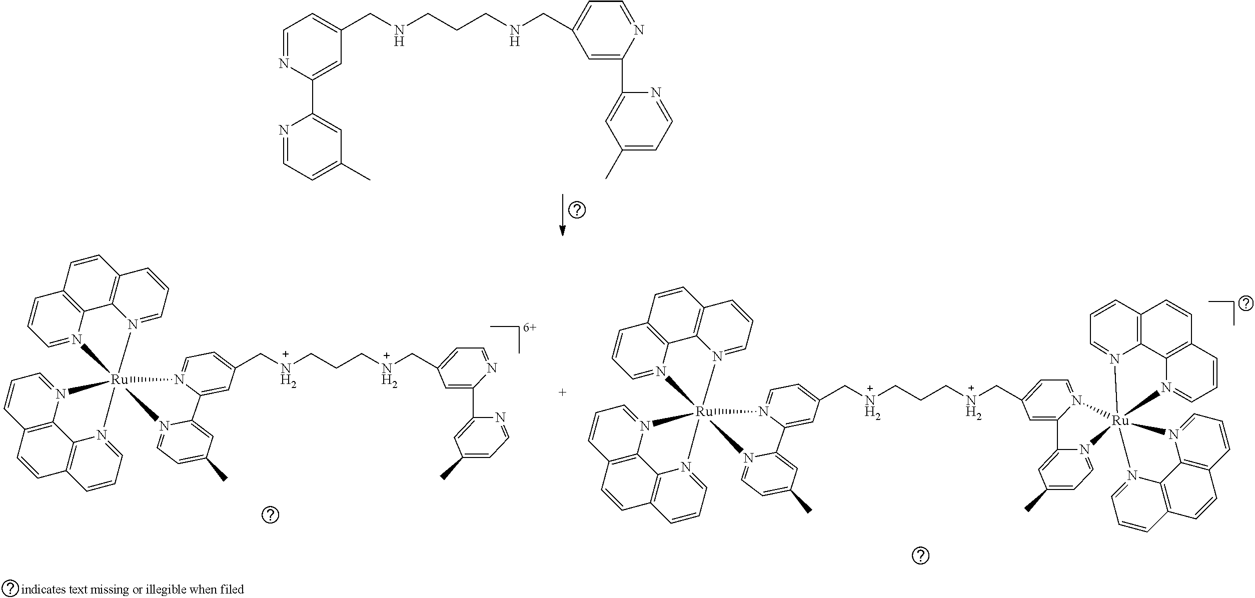

### Synthesis of Mononuclear Complexes

The mononuclear complex Δ-[Ru(phen)₂(bbH₄N₇)]⁶⁺ was obtained as the side product from the purification of dinuclear Δ-[{Ru(phen)₂}₂(μ-bbH₂N₇)]⁶⁺ using the above procedure. The syntheses of mononuclear Δ-[{Ru(phen)₂(Me₂bipy)]²⁺ and Δ-[Ru(phen)₂(bb₇)]²⁺ were also carried out according to the above procedure using excess 4,4′-dimethyl-2,2′-bipyridine and bb₇, while the synthesis of mononuclear Δ-[Ru(phen)₂(bb₁₆)]Cl₂ was modified as follows. A solution of Δ-[Ru(phen)₂(py)₂]{(−)-AsOtart}₂ (180 mg, 0.169 mmol) in ethylene glycol/water (9:1; 20 ml) was added dropwise to a hot solution of bb₁₆ (0.676 mmol) in 2-methoxyethanol (50 ml) over a period of 4 h in the dark under Ar at 115° C. Stirring was continued for further 2 h, after which the solution was cooled to room temperature and the unreacted bb₁₆ was removed by filtration. Water (10 ml) was added to the filtrate and loaded onto an SP-Sephadex C-25 cation exchange column. Washing with water and elution with 0.6 M NaCl solution removed the impurities.

The bright orange product was eluted with a 1 M NaCl solution containing 40% acetone. Solid KPF₆ was added to the eluate and the complex extracted into DCM. The organic layer was washed with water, dried over anhydrous Na₂SO₄, and evaporated to dryness in vacuo to yield a bright red-orange mixture of mononuclear and dinuclear species. The mixture was dissolved in minimal acetone and a saturated solution of tetraethylammonium chloride in acetone was added dropwise until no more precipitation occurred. The dinuclear species precipitated out as its chloride salt, while the mononuclear complex remained in solution.

Both the precipitate and the solution were loaded onto a Sephadex LH-20 column and washed with acetone, resulting in the elution of the PF₆⁻ form of the mononuclear species, while the chloride salt of the dinuclear complex was retained in the column. The dinuclear complex was finally eluted with methanol. Due to low solubility, the conversion of the PF₆⁻ salt of the mononuclear Δ-[Ru(phen)₂(bb₁₆)](PF₆)₂ into Δ-[Ru(phen)₂(bb₁₆)]Cl₂ using Amberlite IRA-400 anion-exchange resin required high dilution and prolonged stirring.

Δ-[Ru(phen)₂(Me₂bipy)](PF₆)₂.H₂O. Anal. Found C, 45.4; H, 2.70: N, 8.5%. Calcd. for C₃₆H₃₀N₆F₁₂OP₂Ru: C, 45.3; H, 3.17; N, 8.8% ¹H NMR (300 MHz, CD₃CN): d 8.68 (2H, d, J=8.0 Hz, H2, H9); 8.57 (2H, d, J=8.0 Hz, H2, H9); 8.42 (2H, s, bipy6); 8.28 (4H, d, J=4.0 Hz, H4, H7); 8.25 (2H, m, bipy3); 7.93-7.81 (4H, m, H5, H6); 7.61-7.50 (4H, m, H3, H8); 7.15 (2H, d=5.0 Hz, bipy5); 2.55 (6H, s, 2×CH₃bipy). CD {λ/nm (Δε/cm⁻¹M⁻¹), Cl⁻ salt in H₂O} 468 (−14), 418 (14), 285 (−147), 268.5 (−187), 260 (208), 218 (27).

Δ-[Ru(phen)₂(bbH₄N₇)](PF₆)₆.3CH₂Cl₂. Anal. Found C, 32.1; H, 2.51: N, 7.1%. Calc. for C₅₄H₅₆N₁₀Cl₆F₃₆P₆Ru: C, 32.0; H, 2.78; N, 6.9% ¹H NMR, (300 MHz, CD₃CN) d 8.69 (2H, dd, J=8.5, 1.0 Hz, H2, H9); 8.58 (2H, dd, J=8.0, 1.0 Hz, H2, H9); 8.47 (4H, s, bipy6); 8.28 (4H, d, J=4.0 Hz, H4, H7); 8.23 (4H, m, bipy3); 7.94-7.81 (4H, m, H5, H6); 7.62-7.52 (4H, m, H3, H8); 7.23 (2H, dd, J=5.5, 1.0 Hz, bipy5′); 7.16 (2H, d, J=5.5, bipy5); 3.94 (4H, s, 2×CH₂bipy); 3.16 (2H, t, NCH₂); 2.85 (2H, t, NCH₂); 2.57 (6H, s, 2×CH₃bipy); 1.80 (2H, m, CH₂). CD {λ/nm (λs/cm⁻¹M⁻¹), Cl⁻ salt in H₂O} 470 (−22), 421.5 (24), 286.5 (−208), 268 (−273), 260 (330), 217 (57).

Δ-[Ru(phen)₂(bb₇)](PF₆)₂.H₂O. Anal. Found C, 52.6; H, 4.01: N, 8.9%. Calc. for C₅₃H₅₀N₈F₁₂OP₂Ru: C, 52.8; H, 4.18; N, 9.3% ¹H NMR, (300 MHz, CD₃CN) d 8.66 (2H, d, J=8.0 Hz, H2, H9); 8.58-8.48 (2H, m, H2, H9); 8.42 (2H, s, bipy6′); 8.38 (2H, s, bipy6), 8.27 (4H, m, H4, H7); 8.22 (4H, m, bipy3), 7.92-7.77 (4H, m, H5, H6); 7.60-7.49 (4H, m, H3, H8); 7.24 (2H, s, bipy5); 7.14 (2H, dd, J=6.0, 1.0 Hz, bipy5); 2.81 (2H, t, CH₂bipy); 2.73 (2H, t, CH₂bipy); 2.55 (3H, s, CH₃bipy); 2.46 (3H, s, CH₃bipy); 1.69-1.38 (10H, m, 5×CH₂). CD {(λ/nm (Δε/cm⁻¹M⁻¹), Cl⁻ salt in H₂O} 469.5 (−17), 421 (15), 285.5 (−152), 269 (−177), 260 (195), 218 (46).

Δ-[Ru(phen)₂(bb₁₂)](PF₆)₂ and Δ-[Ru(phen)₂(bb₁₆)d](PF₆)₂

Solid bb₁₂ (0.26 mmol) was heated at 120° C. in 2-methoxyethanol (30 ml) on a two neck round bottom flask under Argon. A solution of Δ-[Ru(phen)₂(py)₂](−)AsOTart (0.09 mmol) in ethylene glycol/water (9:1, 20 ml) was added dropwise for five hours and the mixture was further heated for two hours. After cooling, water (10 ml) was added and the mixture was loaded onto Sephadex C-25 cation exchange column, eluted with water and then 0.3 M NaCl to remove the impurities. Elution with 1.0 M NaCl containing 5% acetone gave the pure Δ-[Ru(phen)₂(bb₁₂)]²⁺. In the case of Δ-[Ru(phen)₂(bb₁₆)]²⁺, the complex was obtained using 1.0 M NaCl containing 10% acetone. After acetone removal, excess KPF₆ was added causing the precipitation of the PF6 salt of the complex which was then extracted into dichloromethane followed by evaporation to dryness to give the corresponding Δ-[Ru(phen)₂(bbₙ)](PF₆)₂. Typical yield: 61-64%.

Δ-[Ru(phen)₂(bb₁₂)](PF₆)₂ ¹H NMR, (300 MHz, CD₂Cl₂) δ 8.61-8.50 (m); 8.31-8.18 (m); 7.92-7.88 (m); 7.65 (t), 7.51 (t); 7.30 (d), 7.18 (d); 2.85-2.79 (m); 2.59 (s); 2.53 (s); 1.71-1.67 (m); 1.39-1.24 (m). Δ-[Ru(phen)₂(bb₁₆)](PF₆) 2.27 H₂O.acetone. Anal. Found C, 42.1; H, 6.51: N, 5.7%. Calc. for C₆₅H₁₂₆N₈F₁₂O₂₈P₂Ru: C, 42.0; H, 6.83; N, 6.0%. ¹H NMR (300 MHz, CD₃CN) d 8.66 (2H, d, J=8.4 Hz, H2, H9); 8.56 (2H, dd, J=8.0 Hz, H2, H9); 8.43 (2H, s, bipy6′); 8.39 (2H, s, bipy6); 8.26 (4H, dd, J=3.0, 1.0 Hz, H4, H7); 8.23 (4H, m, bipy3); 7.91 (2H, m, H5, H6); 7.82 (2H, m, H5, H6); 7.60-7.50 (4H, m, H3, H8); 7.24 (2H, s, bipy5); 7.14 (2H, d, J=5.0 Hz); 2.80 (2H, t, CH₂bipy); 2.74 (2H, t, CH₂bipy); 2.55 (3H, s, CH₃bipy); 2.44 (3H, s, CH₃bipy); 1.69-1.22 (28H, m, 24×CH₂). CD{λ/nm (Δε/cm⁻¹M⁻¹), Cl⁻ salt in H₂O} 471 (−4), 421.5 (4), 286 (−36), 269.5 (−40), 259.5 (45), 215.5 (6).

The successful isolation of the mononuclear complexes Δ-[Ru(phen)₂(bbₙ)]²⁺ (referred to as “Rubbₙmono”) enabled the synthesis of higher nuclearity complexes due to the availability of the free 2,2′-bipyridine coordination site that can undergo a further reaction with other species in a complex-as-ligand strategy. For example, its reaction with [Ru(phen)Cl₄]⁻ afforded the important precursor Δ-[Ru*(phen)₂(μ-bb₇)Ru(phen)Cl₂]²⁺ {referred to as “Rubb₇dichloro”; only the Ru centre (asterisked) originating from the mononuclear complex containing bb₇ is chiral}, which reacted with another Δ-[Ru(phen)₂(bb₇)]²⁺ to produce the trinuclear complex [{Ru*(phen)₂}(μ-bb₇){Ru(phen)}(μ-bb₇){Ru*(phen)₂}]⁶⁺ (referred to as “Rubb₇trinuclear”; only the two terminal Ru centres are chiral). In addition to microanalytical and NMR characterisation of the trinuclear species, a high-resolution electrospray ionisation mass spectrum of Ru₃[C₁₁₈H₁₀₄N₁₈](PF₆)₆ gave +2, +3, +4 and +5 ions corresponding to five successive losses of PF₆⁻ ions.

Reaction of this same precursor with bb₇ in 2:1 ratio gave the tetranuclear complex [{Ru*(phen)₂}(μ-bb₇){Ru(phen)}-(μ-bb₇){Ru(phen)}(μ-bbⱼ){Ru*(phen)₂}]⁸⁺ (referred to as “Rubb₇tetranuclear”; only the two terminal Ru centres are chiral) as shown below:

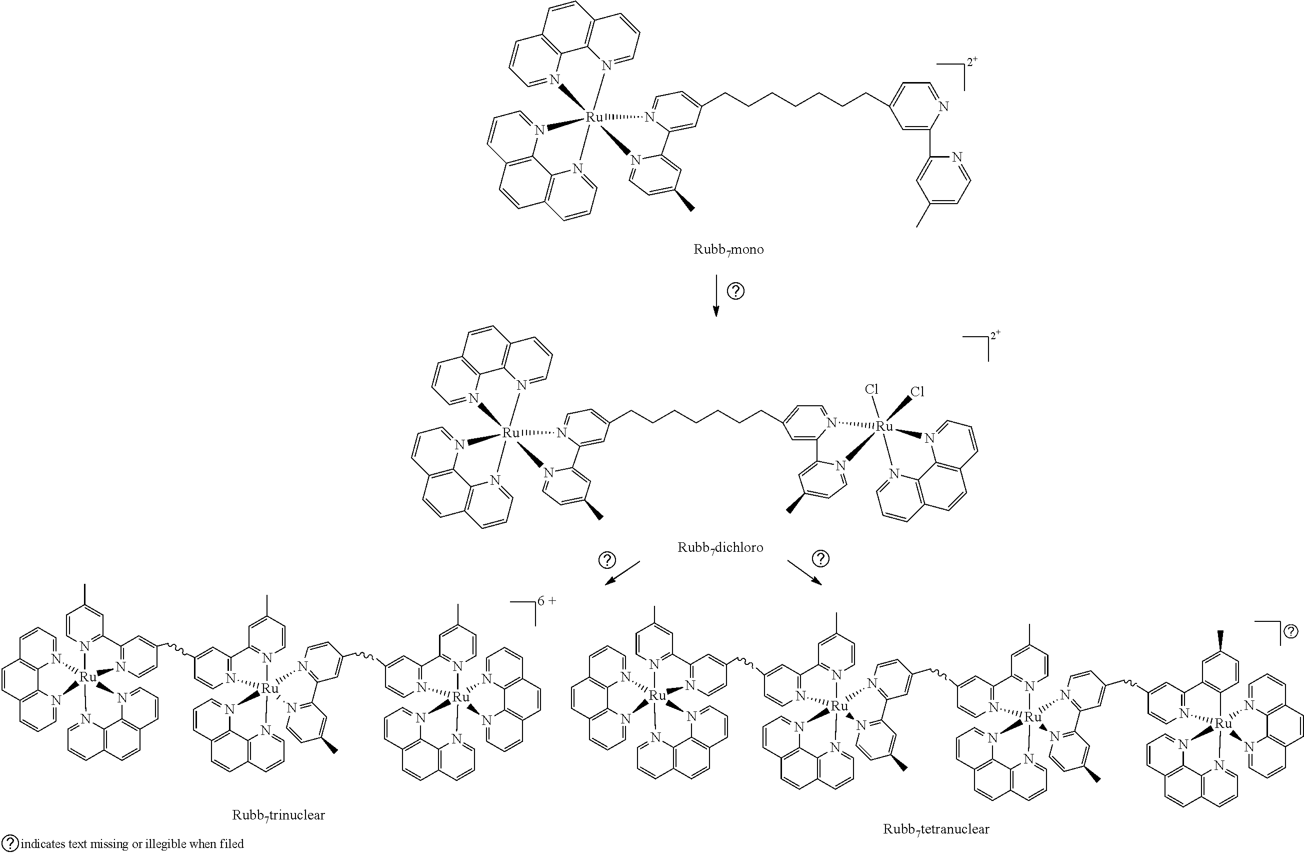

The replacement of the chloro ligands by the 2,2′-bpy moieties was carried out in ethanol water solution under reflux, as reported in T. Tagano, Inorg. Chim. Acta, 1992, 195, 221-225. Characterisation was achieved by microanalysis and NMR methods: despite extensive attempts, the ESI-FTMS determination was unsuccessful for the parent tetranuclear ion, consistent with a sensitivity observed even for the trinuclear species.

Circular dichroism spectra of the trinuclear and tetranuclear complexes were consistent with those for the mononuclear and dinuclear complexes with D chiral metal centres, suggesting that the stereochemistry in both terminal “Ru(phen)₂(bpy)” moieties is maintained. It should be noted that the central “Ru(phen)(bpy)₂” entities may adopt more than one geometric isomeric form, due to the relative orientations of the two Me-bpy and α-CH₂-bpy sites. These stereochemical differences can be observed from the ¹H NMR spectra of both complexes, which show the presence of the multiplet methylene bpy-CH₂-protons (2.82 ppm), in contrast to a typical triplet observed in the (symmetrical) dinuclear complexes. In addition, the presence of the three closely-positioned bpy-CH₃ protons at 2.50, 2.55 and 2.60 ppm further attests to the isomeric complexity.

Synthesis of Dichloro “Precursor Complex” Rubb₇dichloro, Δ*-[{Ru*(phen) 2(μ-bb₇)(Ru(phen)Cl₂}](PF₆)₂ {(only the Ru centre designated by an asterisk is resolved (Δ)}

The synthesis of the complex was adapted from K. Hara, H. Sugihara, L. P. Singh, A. Islam, R. Katoh, M. Yanagida, K. Sayama, S. Murata and H. Arakawa, J. Photochem. Photobiol., A, 2001, 145, 117-122. A typical procedure was as follows. A mixture of Δ-[Ru(phen)₂(bb₇)](PF₆)₂ (80 mg, 0.05 mmol), (phenH⁺)[Ru(phen)Cl₄](40 mg, 0.05 mmol) and lithium chloride (50 mg) in dry DMF (5 ml) was heated to reflux at 150° C. for 8 h in the dark under Ar. Acetone (20 ml) was added to the resulting dark brown solution, causing the precipitation of a dark brown material, which was kept with the mother liquor in the fridge for 12 h. The precipitate was then filtered and washed with acetone and redissolved in ethanol. Solid NH₄PF₆ (50 mg) was added to the solution, resulting in the precipitation of the PF₆⁻ salt of the complex, which was then filtered and washed with ethanol and diethyl ether to afford a dark brown solid. Purification of this product was performed on Sephadex LH-20 using acetone as the eluent. The major first brown band containing Δ-[Ru₂(phen)₃(μ-bb₇)Cl₂](PF₆)₂ was collected, and the product isolated in 20-30% yield. A separation of any possible isomeric products was not attempted.

Δ-[{Ru*(phen)₂(μ-bb₇){Ru(phen)Cl₂}](PF₆)₂.3H₂O. Anal. Found C, 49.0; H, 3.75: N, 8.5%. Calc. for C₆₅H₆₂N₁₀F₁₂O₃P₂Ru₂: C, 49.0; H, 3.92; N, 8.8%. ¹H NMR (300 MHz, CD₂Cl₂) d 8.65-8.58 (3H, m, H2, H9); 8.50 (6H, dd, J=8.0, 1.0, H2, H9); 8.36 (4H, m, bipy6); 8.25-8.17 (10H, m, H4, H7, bipy3); 7.90-7.86 (6H, m, H5, H6); 7.64 (3H, m, H3, H8); 7.49 (3H, m, H3, H8); 7.16 (4H, m, bipy5); 2.73 (4H, m, CH₂bipy); 2.58 (6H, s, 2×CH₃bipy); 1.68-1.40 (10H, m, 5×CH₂). CD{λ/nm (Δε/cm⁻¹M⁻¹), Cl⁻ salt in H₂O} 468 (−11), 420 (12), 286.5 (−112), 268.5 (−136), 259 (160), 216.5 (31).

Synthesis of Trinuclear Complex Rubb₇Trinuclear, Δ*Δ*-[{Ru*(Phen)₂}(μ-bb₇){Ru(phen)}(μ-bb₇){Ru*(phen)₂}](PF₆)₆ {Only the Ru Centres Designated by an Asterisk are Resolved (Δ)}

A mixture of Δ-[Ru₂(phen)₃(μ-bb₇)Cl₂](PF₆)₂ (40 mg, 0.025 mmol) and Δ-[Ru(phen)₂(bb₇)](PF₆)₂ (40 mg, 0.025 mmol) in ethanol-water (1:1.20 ml) was refluxed for three hours in the dark under an Ar atmosphere. The solution slowly turned from brown to dark red during the course of the reaction. The resulting solution was cooled to room temperature and the solvent was evaporated under reduced pressure to obtain a dark orange solid, which was redissolved in a minimum amount of acetone and converted to chloride salt in water using Amberlite IRA-400 anion-exchange resin. After filtration, the solution was loaded onto an SP Sephadex C-25 cation exchange column, washed with water and eluted with 0.3 M NaCl solution to remove mononuclear impurities. The desired trinuclear species was eluted with 10% acetone in 1 M NaCl solution. After removing the acetone, solid KPF₆ was added followed by extraction into dichloromethane. The organic layer was washed with water, dried over anhydrous Na₂SO₄, and evaporated to dryness to yield a bright red-orange precipitate, ΔΔ-[(Ru₃(phen)₅(μ-bb₇)₂](PF₆)₆. The complex was further purified on Sephadex LH-20 using acetone as the eluent. Typical yield 40-50%. A separation of any possible isomeric products was not attempted.

Δ*Δ*-[{Ru*(phen)₂}(μ-bb₇){Ru(phen)}(μ-bb₇){Ru*(phen)₂}]-(PF₆)₆.2H₂.O₂acetone. Anal: Found C, 48.0; H, 3.75: N, 8.0%. Calcd. for C₁₂₄H₁₂₀N₁₈F₃₆O₄P₆Ru₃: C, 48.0; H, 3.90; N, 8.1%. ¹H NMR (300 MHz, CD₃CN) d 8.69-8.56 (10H, m, H2, H9); 8.44-8.31 (8H, m, bipy6); 8.27 (10H, m, H4, H7); 8.23 (8H, m, bipy3); 7.92 (5H, m, H5, H6); 7.86-7.68 (5H, m, H5, H6); 7.61-7.51 (10H, m, H3, H8); 7.34 (4H, m, bipy5′); 7.12 (4H, m, bipy5); 2.82 (8H, m, CH₂bipy; 2.60 (3H, s, CH₃bipy); 2.55 (6H, s, 2×CH₃bipy); 2.50 (3H, s, CH₃bipy); 1.72-1.38 (20H, m, 10×CH₂). CD{λ/nm (Δε/cm⁻¹M⁻¹), Cl⁻ salt in H₂O} 470 (−22), 419.5 (21), 286.5 (−215), 270 (−235), 258.5 (280), 217.5 (69). FTMS (+ESI): most abundant ion m/z 837.4985 ([M-3PF₆]³⁺) calc. for Ru₃[C₁₈H₁₀₄N₁₈](PF₆)₃³⁺, 837.4929; most abundant ion m/z 1328.7241 ([M-2 PF₆]²⁺) calc. for Ru₃[C₁₁₈H₁₀₄N₁₈](PF₆)₄²⁺, 1328.7218.

Synthesis of Tetranuclear Complex Rubb₇Tetranuclear, Δ*Δ*-[{Ru*(Phen)₂}(μ-bb₇){Ru(phen)}(μ-bb₇){Ru*(phen)}(μ-bb₇){Ru*(phen)₂}](PF₆)₆ {Only the Ru Centres Designated by an Asterisk are Resolved (Δ)}

A mixture of Δ-[Ru₂(phen)₃(μ-bb₇)(Cl)₂](PF₆)₂ (40 mg, 0.025 mmol) and bb₇ (5 mg, 0.013 mmol) was heated to reflux in ethanol-water (1:1.20 ml) for 3 h in the dark under Ar atmosphere. The solution slowly turned from brown to dark red during the course of the reaction. The resulting solution was cooled to room temperature and the solvent was evaporated under reduced pressure to obtain a dark orange solid, which was redissolved in acetone (2 ml) followed by a dropwise addition of a saturated solution of tetraethylammonium chloride, causing the precipitation of its chloride salt. After the acetone removal, the solid was dissolved in water (5 ml) and loaded onto an SP-Sephadex C-25 cation exchange column, washed with water and eluted with 0.3 M NaCl solution to remove the impurities. The desired tetranuclear species was eluted with a 1 M NaCl solution containing 10% acetone. After removing the acetone, solid KPF₆ was added followed by extraction into DCM. The organic layer was washed with water, dried over anhydrous Na₂SO₄, and evaporated to dryness to yield a bright red-orange precipitate, ΔΔ-[(Ru₄(phen)₆(μ-bb₇)₃](PF₆)₈. The complex was further purified using Sephadex LH-20 with acetone as the eluent. Typical yield 60%. A separation of any possible isomeric products was not attempted.

Δ*Δ*-[{Ru*(phen)₂}(μ-bb₇) {Ru(phen)₂}(μ-bb₇) {Ru(phen)₂}(μ-bb₇) {Ru*(phen)₂}](PF₆).8H₂O Anal. Found C, 48.1; H, 3.63: N, 8.3%. Calcd. for C₁₅₉H₁₄₆N₂₄F₄₈OP₈Ru₄: C, 48.1; H, 3.70; N, 8.5%. ¹H NMR (300 MHz, CD₃CN) d 8.70-8.57 (12H, m, H2, H9); 8.45-8.35 (12H, m, bipy6); 8.27 (12H, m, H4, H7); 8.25 (12H, m, bipy3); 7.92 (6H, m, H5, H6); 7.85-7.64 (6H, m, H5, H6); 7.60-7.52 (12H, m, H3, H8); 7.34 (6H, m, bipy5′); 7.12 (6H, m, bipy5); 2.82 (8H, m, CH₂bipy); 2.60 (3H, s, CH₃bipy); 2.56 (12H, s, CH₃bipy); 2.51 (3H, s, CH₃bipy); 1.70-1.41 (30H, m, 15×CH₂). CD{λ/nm (Δε/cm⁻¹M⁻¹), Cl⁻ salt in H₂O} 470.5 (−26), 417.5 (25), 289 (−255), 268 (−266), 258 (293), 218 (58).

Representative synthesis of [{Ru(terpy)Cl}₂(μ-bbₙ)]²⁺ (n=7, 10, 12 and 14)

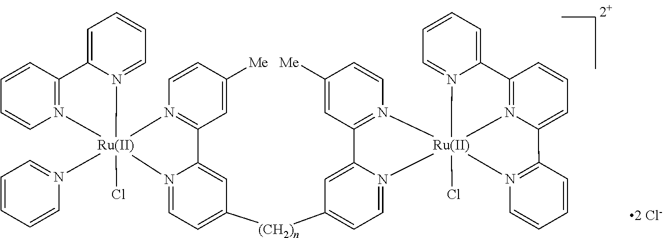

As used herein these complexes are also referred to by shorthand notation. For example [{Ru(terpy)Cl}₂(μ-bb₇)] is referred to as Rubb₇-Cl.

Solid [Ru(terpy)Cl₃](0.20 g, 0.45 mmol) and bb, (0.23 mmol) were refluxed in EtOH/H₂O (4:1; 40 ml) for 4 h. After cooling, the solvent mixture was evaporated to approximately half of the original volume and excess NH₄ PF₆ was added causing the precipitation of the dark brown-purple material, which was filtered and washed with cold ethanol followed by diethyl ether. The crude product was dissolved in acetone and loaded onto a Sephadex LH-exclusion column, and separated using acetone as the eluent. The pure dinuclear [{Ru(terpy)(Cl)}₂(μ-bbₙ)](PF₆)₂ complexes were isolated as dark purple materials. The chloride salts were obtained by stirring the solid in an aqueous solution using Amberlite® IRA-400 (chloride form) anion-exchange resin. The resin was removed by filtration, and the dark purple solution was freeze-dried to obtain a fluffy dark red purple powder of [{Ru(terpy)(Cl)}₂(μ-bbₙ)]Cl₂. Typical yields after conversion: 30-60%. Separation of any possible geometric isomers of [{Ru(terpy)(Cl)}₂(μ-bbn)]²⁺ was not attempted.

[{Ru(terpy)(Cl)}₂(μ-bb₇)](PF₆)₂.3H₂O: Anal. Found C, 46.8; H, 3.63: N, 8.7%. Calcd, for C₅₉H₆₀N₁₀F₁₂O₃P₂Ru₂: C, 46.6; H, 3.98; N, 9.2% ¹H NMR (300 MHz, CD₃CN) d 10.04 (s); 8.61 (s); 8.49 (m); 8.40 (d, J=7.2 Hz); 8.31 (d, J=7.2 Hz); 8.20 (d, J=11.7 Hz); 8.10 (t); 7.90 (t); 7.81 (s); 7.70 (s); 7.55 (s); 7.30 (s); 7.12 (t); 6.81 (d, 5.1 Hz); 3.04 (t); 2.84 (t); 2.78 (s); 2.77-2.55 (m); 1.76 (s); 1.53 (s); 1.37-1.22 (m).

[{Ru(terpy)(Cl)}₂(μ-bb₁₀)](PF₆)₂.3acetone: Anal. Found C, 50.7; H, 4.49: N, 8.4%. Calcd. for C₇₁H₇₈N₁₀F₁₂O₃P₂Ru₂: C, 50.7; H, 4.67; N, 8.3% ¹H NMR (300 MHz, CD₃CN) d 10.04 (s); 8.64 (s); 8.51 (d, J=8.1 Hz); 8.40 (d, J=7.8 Hz); 8.33 (m); 8.22 (m); 8.10 (t); 7.91 (t); 7.81 (s); 7.71 (s); 7.60 (s); 7.30 (s); 7.12 (t); 6.80 (s); 3.04 (t); 2.85 (t); 2.78 (s); 2.60 (s); 1.75 (s); 1.50 (s); 1.41-1.22 (m).

[{Ru(terpy)(Cl)}₂(μ-bb₁₂)](PF₆)₂.5acetone: Anal. Found C, 52.5; H, 4.69: N, 8.5%. Calcd. For C₇₉H₉₄N₁₀F₁₂O₅P₂Ru₂: C, 52.0; H, 5.19; N, 7.8% ¹H NMR (300 MHz, CD₃CN) d 10.04 (s); 8.65 (s); 8.50 (m); 8.47-8.34 (m); 8.20 (d, J=12.0 Hz); 8.09 (t); 7.90 (t); 7.82 (s); 7.71 (s); 7.30 (s); 7.12 (t); 6.81 (d, 5.7 Hz); 3.04 (t); 2.89 (t); 2.78 (s); 2.63-2.55 (m); 1.76 (s); 1.53 (s); 1.37-1.22 (m).

[{Ru(terpy)(Cl)}₂(μ-bb₁₄)](PF₆)₂.3acetone: Anal. Found C, 52.1; H, 4.88: N, 8.5%. Calcd. for C₇₅H₈₆N₁₀F₁₂O₃P₂Ru₂: C, 51.8; H, 4.99; N, 8.1% ¹H NMR (300 MHz, CD₃CN) d 10.04 (s); 8.61 (s); 8.49 (m); 8.40 (d, J=7.2 Hz); 8.31 (d, J=7.2 Hz); 8.20 (d, J=11.7 Hz); 8.10 (t); 7.90 (t); 7.81 (s); 7.70 (s); 7.55 (s); 7.30 (s); 7.12 (t); 6.81 (d, 5.1 Hz); 3.04 (t); 2.84 (t); 2.78 (s); 2.77-2.55 (m); 1.76 (s); 1.53 (s); 1.37-1.22 (m).

Representative Synthesis of [Ru(phen)₂(μ-bbₙ)Ru(terpy)Cl]Cl₃

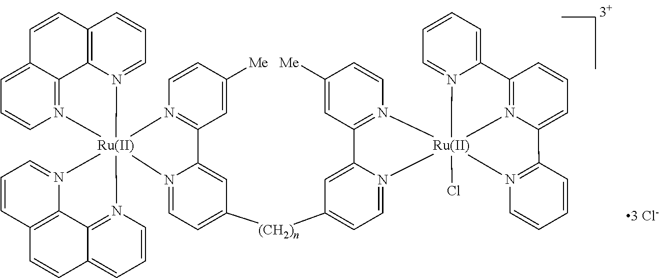

As used herein these complexes are also referred to by shorthand notation. For example [Ru(phen)₂(μ-bb₇)Ru(terpy)Cl] is referred to as Ru(μ-bb₇)-RuC.

Solid [Ru(terpy)Cl₃] (0.032 mmol) and Δ-[Ru(phen)₂(bbₙ)](PF₆)₂ (0.032 mmol) were refluxed in ethanol/water (4:1, 10 ml) for three hours. After cooling, excess amount of NH₄ PF₆ was added causing the precipitation of a dark brown material which was filtered and washed with ethanol. The brown crude was then loaded onto Sephadex LH20 exclusion chromatography using acetone as the eluent. The [Ru(phen)₂(μ-bbₙ)Ru(terpy)Cl](PF₆)₃ fraction was obtained as the major dark brown band which was isolated and evaporated to dryness. The PF₆⁻ salt was converted to the chloride by dissolving the solid in the minimum amount of acetone followed by addition of the saturated solution of tetraethylammonium chloride in acetone drop wise while stirring for half an hour. The resulting fluffy precipitates were centrifuged, decanted, washed several times with cold acetone and dried under reduced pressure to afford [Ru(phen)₂(Δ-bbₙ)Ru(terpy)Cl](Cl)₃ in 70-80% yield. Separation of any possible geometric isomers was not attempted.

[Ru(phen)₂(μ-bb₇)Ru(terpy)Cl](PF₆)₃ ¹H NMR (300 MHz, CD₂Cl₂) δ 10.13 (s); 8.63 (d); 8.53 (d); 8.43-8.35 (m); 8.27-8.11 (m), 8.01-7.62 (m); 7.55-7.45 (m), 7.26-7.15 (m); 7.00 (t); 6.78 (t); 3.01-2.79 (m); 2.64-2.59 (m); 2.38 (s); 1.69-1.26 (m).

[Ru(phen)₂(μ-bb₁₂)Ru(terpy)Cl](PF₆)₃ ¹H NMR (300 MHz, CD₂Cl₂) δ 10.22 (s); 8.64 (d); 8.53 (d); 8.43-8.35 (m); 8.34-8.09 (m), 8.91-7.87 (m); 7.76-7.65 (m), 7.53-7.49 (m); 7.35 (s); 7.18 (s); 6.89 (s); 6.81 (s); 3.24 (t); 3.08 (t); 2.84 (s); 2.76-2.70 (m); 2.59 (s); 2.55 (s), 1.74-1.30 (m).

[Ru(phen)₂(μ-bb₁₆)Ru(terpy)Cl](PF₆)₃ ¹H NMR (300 MHz, CD₂Cl₂) δ 10.25 (s); 8.63 (d); 8.52 (d); 8.37-8.17 (m); 8.07 (d), 7.91-7.66 (m); 7.51-7.47 (m), 7.32-7.19 (m); 6.99 (d); 6.81 (d); 3.07 (t); 2.86 (t); 2.66-2.59 (m); 2.43 (s); 1.60-1.20 (m).

### Minimum Inhibitory Concentration (MIC) Determination

Note: The bacterial strains used in this study are classified as risk group 2 according to the Australian/New Zealand Standard (AS/NZS 2243.4:2010) and must be manipulated in a PC2 class laboratory.

Four strains of bacteria were chosen for the susceptibility tests: S. aureus ATCC 25923; MRSA (a wild clinical strain from the JCU culture collection); E. coli ATCC 25922 and P. aeruginosa ATCC 27853. The bacteria were grown on Mueller Hinton agar (OXOID, cat. No. CM0337) and suspended in growth medium cation-adjusted Mueller Hinton broth (CAMHB, OXOID, cat. No. CM0405). The MIC of each complex was determined in duplicate by standard micro-dilution methodology (British Society for Antimicrobial Chemotherapy Working Party, J. Antimicrob. Chemother., 1991, 27(suppl. D), 22) using gentamicin as the positive control. The ruthenium complexes were dissolved in CAMHB to a stock solution of 512 mg ml⁻¹ and two-fold diluted in the broth in 96-well plates in a final volume of 100 ml in each well. The same volume (100 ml) of bacterial suspension was added in each well making a concentration range of 0.25 mg ml⁻¹ to 128 mg ml⁻¹ for each complex. The plates were incubated at 37° C. for 24 h. The results are summarised in Table 1 below, using the following abbreviations:
Rubpm=ΔΔ-[{Ru(phen)₂}₂(μ-2,2′-bipyrimidine)]Cl₄
Rudppm=ΔΔ-[{Ru(phen)₂}₂(μ-4,6-bis(2-pyridyl)pyrimidine)]Cl₄
Rubb₇trinuclear=Δ*Δ*-[{Ru*(phen)₂}(μ-bb₇){Ru(phen)}(μ-bb₇){Ru*(phen)₂}]Cl₆ (only the two terminal metal centres are chiral, the two central metal centres are racemic)
Rubb₇tetranuclear=Δ*Δ*-[{Ru*(phen)₂}(μ-bb₇) {Ru(phen)}(μ-bb₇){Ru(phen)}(μ-bb₇)-{Ru*(phen)₂}]Cl₈ (only the two terminal metal centres are chiral, the two central metal centres are racemic)

The minimum inhibitory concentration (MIC) results for the ruthenium complexes against four bacterial strains shown in Table 1 demonstrate that some of the ruthenium(II) complexes are highly active against both classes of bacteria, although Gram-positive bacteria appeared to be more susceptible than the Gram-negative counterparts. Of particular note are the low MIC values of the ΔΔ/ΛΛ enantiomers of Rubb₁₂, Rubb₁₄ and Rubb₁₆ against both S. aureus and the drug-resistant strain MRSA (an MIC of 1 μg mL⁻¹ is equivalent to 0.5 μM). Only slight differences in activity were observed between the ΔΔ- and ΛΛ-enantiomers of Rubb₇, Rubb₁₂, Rubb₁₄ and Rubb₁₆, suggesting that chiral receptors—such as proteins or nucleic acids—may not be the main intracellular target for these metal complexes. Interestingly, the dinuclear complexes with a short linking chain (bb₂ and bb₅), a rigid polycyclic aromatic linking ligand (bpm and dppm) or those containing an ether or amine in the linking ligand, showed very little or no observable activity against any of the bacterial strains. The trinuclear and tetranuclear analogues of ΔΔ-Rubb₇ showed slightly better activity than the dinuclear complex. The highly lipophilic mononuclear complexes (as determined by octanol-water partition coefficient−logP values) containing the bb₇ or bb₁₆ ligand exhibited intermediate MIC values, although Rubb₇mono was more active (on a mole basis) against S. aureus than its dinuclear counterpart.

The mononuclear complex [Ru(phen)₃]²⁺ exhibited no activity; however, the incorporation of methyl groups on the phenanthroline ligands significantly increased the activity against all strains. [Ru(Me₄phen)₃]²⁺ showed equal activity (on a mole basis) to ΔΔ/ΛΛ Rubb₁₂, Rubb₁₄ and Rubb₁₆ against S. aureus, but was less active against the drug-resistant strain MRSA. This indicates that MRSA is resistant to the mononuclear complexes but not the dinuclear complexes. It is possible that the dinuclear complexes could just pass through the cell wall and/or the cytoplasmic membrane more easily, or alternatively the mechanisms of resistance are not effective against the dinuclear complexes.

TABLE 1
In vitro antimicrobial activity MIC (minimum inhibitory concentration)
of ruthenium complexes against four bacterial strains

| | | MIC (μg.ml⁻¹) | | | |
|---|---|---|---|---|---|
| | | Gram-positive | | Gram-negative | |
| | | S. | | E. | P. |
| Group | Compound | aureus | MRSA | coli | aeruginosa |
| Rigid link | Rubpm | >128 | >128 | >128 | >128 |
| dinuclear | Rudppm | >128 | >128 | >128 | >128 |
| Flexible | ΛΛ-Rubb₂ | >128 | >128 | | >128 |
| link | ΔΔ-Rubb₂ | >128 | >128 | >128 | >128 |
| dinuclear | ΛΛ-Rubb₅ | 128 | 128 | 64 | 128 |
| | ΔΔ-Rubb₅ | 128 | 128 | 128 | >128 |
| | ΛΛ-Rubb₇ | 64 | 32 | 16 | 64 |
| | ΔΔ-Rubb₇ | 16 | 32 | 16 | 128 |
| | ΛΛ-Rubb₁₀ | 8 | 8 | 4 | 32 |
| | ΔΔ-Rubb₁₀ | 4 | 4 | 4 | 64 |
| | ΛΛ-Rubb₁₂ | 2 | 2 | 2 | 16 |
| | ΔΔ-Rubb₁₂ | 1 | 1 | 2 | 16 |
| | ΛΛ-Rubb₁₄ | 1 | 1 | 2 | 8 |
| | ΔΔ-Ruhh₁₄ | 1 | 1 | 4 | 8 |
| | ΛΛ-Rubb₁₆ | 1 | 1 | 4 | 8 |
| | ΔΔ-Rubb₁₆ | 1 | 1 | 4 | 8 |
| Flexible | ΔΔ-RubbN₇ | >128 | >128 | >128 | >128 |
| link | ΔΔ-RubbO₇ | 128 | 128 | 128 | >128 |
| dinuclear | ΔΔ-RubbN₁₂ | 128 | 128 | 128 | >128 |
| with | | | | | |
| N, O in | | | | | |
| the chain | | | | | |
| Mono- | [Ru(phen)₂ | 128 | >128 | >128 | >128 |
| nuclear | (Me₂bpy)]²⁺ | | | | |
| with bbₙ | Rubb₇mono | 4 | 16 | 16 | 32 |
| ligands | Rubb₁₆mono | 16 | 16 | 64 | 64 |
| Mono- | [Ru(phen)₃]²⁺ | >128 | >128 | >128 | >128 |
| nuclear | [Ru(Me₂phen)₃]²⁺ | 8 | 128 | 128 | >128 |
| with phen | [Ru(Me₄phen)₃]²⁺ | 0.5 | 4 | 8 | 32 |
| or | | | | | |
| Meₙphen | | | | | |
| ligands | | | | | |
| Tri- and | Rubb₇trinuclear | 4 | 4 | 16 | 2 |
| tetra- | Rubb₇tetranuclear | 8 | 8 | 32 | 8 |
| nuclear | | | | | |
| Control | Gentamicin | ≦0.0625 | 16 | 0.5 | 0.125 |

It has been shown that some Gram-positive bacteria, such as S. aureus, develop resistance to cationic antibiotics by altering the structure of the teichoic acids in the cell wall in order to decrease the negative charge on the cell surface (A. Peschel, M. Otto, R. W. Jack, H. Kalbacher, G. Jung and F. Gotz, J. Biol. Chem., 1999, 274, 8405). MRSA strains with reduced negatively-charged teichoic acids will exhibit lower electrostatic attraction to cationic antimicrobial molecules on the cellwall and membrane. Consistent with this proposal, the positive control drug used in this study—gentamicin (an aminoglycoside)—is positively charged at neutral pH and it was considerably less active against MRSA than the susceptible ATCC 25923 S. aureus. Similarly, mononuclear ruthenium complexes with a +2 charge may be less effective against MRSA than the susceptible strain. By contrast, the dinuclear Rubbₙ complexes, which have a higher charge (14) that is also spread over two metal centres, exhibited equal activity against both strains. It was also noted that the tri- and tetranuclear complexes exhibited equal activity against S. aureus and MRSA. The results also suggest that a trinuclear complex based upon either a bb₁₂, bb₁₄ or bb₁₆ ligand could be even more effective than their dinuclear counterparts.

The primary difference between the members of the Rubbₙ family of complexes is the number of methylene groups in the flexible linking chain. The observed differences in the MIC values of the complexes therefore suggest that the antibacterial activity is correlated with the lipophilicity of the metal complex. Lipophilicity is a significant factor affecting the biological activity of most therapeutic compounds, as it relates to the capacity of the drug to penetrate through the cell membrane. The standard octanol-water partition coefficient (logP) was determined for the Rubbₙ series (F. Lombardo, B. Faller, M. Shalaeva, I. Tetko and S. Tilton, Methods and Principles in Medicinal Chemistry, 2008, 37, 407) parent model mononuclear species ([Ru(phen)₂(Me₂bpy)]²⁺) and the trinuclear and tetranuclear complexes.

The partition coefficients (logP) were measured using the shake flask technique: each ruthenium complex (at both 0.05 and 0.1 mM) was dissolved in the water phase (0.2 M Na₂HPO₄—NaH₂PO₄ buffer, pH=7.4) and an equivalent volume of n-octanol was added.

The two phases were mutually saturated by shaking overnight at ambient temperature and then were allowed to separate on standing. The concentration of the metal complex in each phase was determined spectrophotometrically at l=450 nm. The results are summarised in Table 2 below:

TABLE 2
The octanol-water partition coefficient (logP) values

| Metal complex | Charge | logP |
|---|---|---|
| Rubb₂ | +4 | −3.6 |
| Rubb₅ | +4 | −3.5 |
| Rubb₇ | +4 | −3.4 |
| Rubb₁₀ | +4 | −3.3 |
| Rubb₁₂ | +4 | −2.7 |
| Rubb₁₄ | +4 | −2.3 |
| Rubb₁₆ | +4 | −1.9 |
| Rubb₇trinuclear | +6 | −1.2 |
| Rubb₇tetranuclear | +8 | −1.9 |
| [Ru(phen)₂(Me₂bpy)]²⁺ | +2 | −2.9 |
| Rubb₇mono^{a} | +2 | −0.7 |
| Rubb₁₆mono^{a} | +2 | +2.1 |

^{a}As the Rubb₇mono and Rubb₁₆mono complexes contain ionisable bpy-N groups, logD should be used (F. Lombardo, B. Faller, M. Shalaeva, I. Tetko and S. Tilton, Methods and Principles in Medicinal Chemistry, 2008, 37, 407). However the experimental pH 7.4 > pKa (5.45), (B. R. James and R. J. P. Williams, J. Chem. Soc., 1961, 2007) and therefore, logD = logP. (B. R. James and R. J. P. Williams, J. Chem. Soc., 1961, 2007)

For the dinuclear Rubbₙ complexes, it is apparent that the activity is related to the lipophilicity as measured by logP. However, it is also noted that the highly lipophilic mononuclear complex Rubb₁₆mono was considerably less active than ΔΔ/ΛΛ Rubb₁₄ and Rubb₁₆. This observation indicates that the overall complex charge is important as well as the lipophilicity, and further highlights the greater antibacterial potential of the oligonuclear species compared to the mononuclear complexes. The logP values do not change in a consistent linear manner along the series from Rubb₂ to Rubb₁₆, with ΔlogP/CH₂ greater for Rubb₁₀-Rubb₁₆ than for Rubb₂-Rubb₁₀. Analysis of the dinuclear complexes by NMR spectroscopy indicates that the ruthenium metal centres can fold back upon themselves (intramolecular folding), with the maximum folding occurring for Rubb₁₀. Consequently, from Rubb₂ to Rubb₁₀ the increasing octanol solubility arising from the increasing number of methylene groups is partially offset by the intramolecular folding. On the other hand, for Rubb₁₂ to Rubb₁₆ the lipophilicity increases with the number of methylene groups and the unfolding of the linking chain.

Further MIC assays were conducted against five different bacteria with Rubbₙ (n=7, 10, 12, 16), two rigid dinuclear species (bridges bpm and dppm) and Gentamicin—Whole spectrum bacteria MIC test results (Broth: CAMHB). The results of the further MIC assays are shown in Table 3 below:

TABLE 3
MIC assays against five different bacteria

| | Gram-positive | | | Gram-negative | |
|---|---|---|---|---|---|
| | S. | E. | | S. | A. |
| | epidermidis | faecalis | VRE | typhimurium | baumanii |
| ΔΔ-Rubb₇ | 1 | 32 | 16 | >128 | 64 |
| ΔΔ-Rubb₁₀ | 0.5 | 8 | 4 | 32 | 64 |
| ΔΔ-Rubb₁₂ | 0.5 | 4 | 2 | 8 | 8 |
| ΔΔ-Rubb₁₆ | 1 | 2 | 2 | 8 | 8 |
| Rubpm | >128 | >128 | >128 | >128 | >128 |
| Rudppm | >128 | >128 | >128 | >128 | >128 |
| Gentamicin | <0.125 | 32 | 16 | 2 | 16 |

The results shown in table 3 indicate that the dinuclear complexes of the present invention containing flexible linking groups (Q) were as active, or significantly more active, against a number of the bacterial strains tested than the control gentamicin.

Further MIC assays and Minimum Bactericidal Concentration (MBC) assays were conducted against Gram-positive and Gram-negative bacterial strains using symmetric and non-symmetric, inert and labile complexes. The results are summarised in Table 4 below:

TABLE 4
MICs of three groups of dinuclear ruthenium complexes (all ΔΔ- enantiomers)

| | | Gram-positive | | | | Gram-negative | | | |
|---|---|---|---|---|---|---|---|---|---|
| Broth | | S. aureus | | MRSA | | E. coli | | P. aeruginosa | |
| (CAMHB) | | MIC | MBC | MIC | MBC | MIC | MBC | MIC | MBC |
| Symmetric | Rubb₇ | 16 | 32 | 16 | 32 | 16 | 16 | 64 | 128 |
| Inert | Rubb₁₂ | 1 | 2 | 1 | 2 | 2 | 2 | 16 | 32 |
| dinuclear | Rubb₁₆ | 1 | 1 | 1 | 2 | 2 | 2 | 8 | 8 |
| Symmetric | Rubb₇-Cl | 8 | 8 | 8 | 16 | 8 | 8 | 32 | 64 |
| labile | Rubb₁₂-Cl | 1 | 1 | 1 | 1 | 2 | 2 | 8 | 16 |
| dinuclear | Rubb₁₄-Cl | 1 | 1 | 1 | 2 | 2 | 2 | 32 | 64 |
| | Rubb₁₆-Cl | 8 | 8 | 8 | 8 | 8 | 8 | >128 | >128 |
| Non- | Ru(μ-bb₇)Ru—Cl | 8 | 16 | 8 | 32 | 1 | 1 | 16 | 32 |
| symmetric | Ru(μ-bb₁₂)Ru—Cl | 1 | 1 | 1 | 2 | 1 | 1 | 16 | 32 |
| dinuclear | Ru(μ-bb₁₆)Ru—Cl | 2 | 2 | 2 | 2 | 4 | 4 | 64 | 64 |
| (one side | | | | | | | | | |
| inert, one | | | | | | | | | |
| side labile) | | | | | | | | | |

The results appear to suggest that both the MIC and MBC activities increased against Gram-positive and Gram-negative strains up to an alkylene linker group length of approximately C₁₂ for both the symmetric inert dinuclear ruthenium complexes and the symmetric labile dinuclear ruthenium chloro complexes. The activity trend appeared to be matched for the non-symmetric dinuclear ruthenium chloro complexes against Gram-positive strains whereas the activity of the non-symmetric dinuclear ruthenium chloro complexes against Gram-negative strains remained approximately the same for alkylene linker group lengths of C₇₋₁₂ and became worse as the size increased beyond C₁₂.

The results appear to suggest that for Gram-positive strains all the dinuclear complexes were approximately equally effective against the susceptible and the resistant strain. When n=12 the complexes of the three groups showed similar activity, and the complexes with the labile ligands showed better activity than their inert counterparts when n<12 while became less effective when n>12. Without wishing to be bound by theory, this observation might be due to the labile ligand affecting the uptake rate and amount of the complexes and/or the DNA attraction inside the cells weakened the interaction of the main target the cell membrane.

For Gram-negative strains, the three groups of complexes showed quite different patterns but the complexes with n=12 were the most active in every group. Ru(μ-bb₇)Ru—Cl and Ru(μ-bb₁₂)Ru—Cl were the most active complexes against E. coli.

All the complexes tested are considered bactericidal, having MBC/MIC≦2. The Ru(μ-bb₁₂)Ru—Cl complex showed the best activity against the four bacteria strains among all the compounds tested.

The variations of MIC values are possibly related to the lipophilicity of these complexes.

Rubb₁₆ is considerably more lipophilic than Rubb₁₂ (almost one log 10 unit), and the non-symmetric complexes are more lipophilic than the inert dinuclear. The symmetric labile complexes are the most lipophilic.

It would appear that for Gram-positive, the best degree of lipophilicity occurs with the Rubb₁₂ symmetric labile complex. Rubb₁₄ symmetric labile complex appears to be slightly worse than the equivalent Rubb₁₂ symmetric labile complex.

### Haemolytic Assay

While excellent antibacterial activity is typically essential for a potential new drug, the compound should ideally also exhibit low toxicity towards human or animal eukaryotic cells. As the ruthenium complexes are water-soluble and could potentially be distributed throughout the body in the blood, the haemolytic activity was determined for the dinuclear complexes that showed the best activity (ΔΔ-Rubb₇, ΔΔ-Rubb₁₀, ΔΔ-Rubb₁₂ and ΔΔ-Rubb₁₆) in the antibacterial assays.

Freshly-drawn human blood was collected using lithium heparin vacuum blood collection tubes (Vacutainer, BD Australia). Erythrocytes were separated by centrifugation for 10 min at 1000 g at 10° C. and washed six times with phosphate-buffered saline (PBS) solution and diluted with PBS to a 10% solution. The concentration of erythrocytes was determined by haemocrit centrifugation (Biofuge Haemo, Heraeus instruments). A 1% Triton-X solution was prepared as the positive control. The complexes to be tested were dissolved in PBS to a stock solution of 2048 mg ml⁻¹ and two-fold diluted in PBS in round-bottomed 96-well plates in a final volume of 100 ml in each well. 100 ml red blood cell suspension samples were added to the wells making a concentration range from 1 mg ml⁻¹ to 512 μg ml⁻¹ for Rubbₙ (n=10, 12, 16) and 2 μg ml⁻¹ to 1024 μg ml⁻¹ for Rubb7, and the plates with each complex were respectively incubated for 1, 2, 4, 8 and 24 h at 37° C. After incubation, the plates were centrifuged at 1000 g for 4 min. 100 ml of supernatant in each well was transferred to a corresponding well in a new 96-well microtitre plate. The absorbance of each well was measured at 540 nm using a plate reader (Mutiskan Ascent, Thermo). Each complex was analysed in duplicate. The solutions of the metal complex at the same tested concentrations without erythrocytes were measured as a negative control.

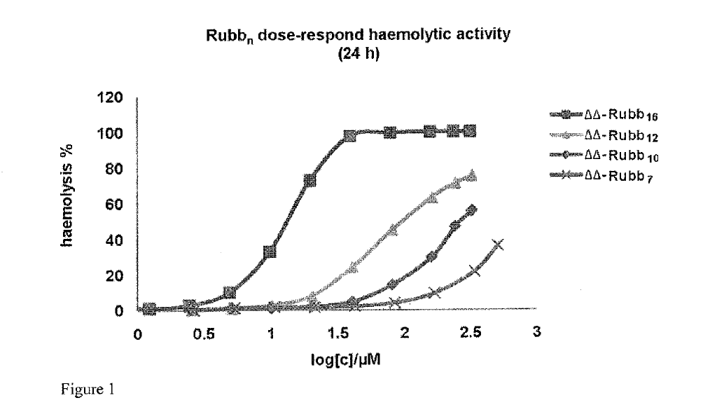

The extent of haemolysis induced by incubation of the ruthenium complexes for 24 h with freshly-collected human red blood cells is shown in FIG. 1 and summarised in Table 5 below:

TABLE 5
HC₅₀ values of four Rubbₙ complexes (ΔΔ-Rubb₇, ΔΔ-Rubb₁₀, ΔΔ-Rubb₁₂ and
ΔΔ-Rubb₁₆) after 24 h incubation with red blood cells

| | | | First indication (>2%) |
|---|---|---|---|
| Complex | HC₅₀ (μM) | HC₅₀ (mg ml⁻¹) | of haemolysis (μg.ml⁻¹) |
| ΔΔ-Rubb₇ | >682 | >1024 | 64 |
| ΔΔ-Rubb₁₀ | 265.6 | 410 | 32 |
| ΔΔ-Robb₁₂ | 101.8 | 160 | 16 |
| ΔΔ-Robb₁₆ | 13.5 | 22 | 4 |

HC₅₀: concentration needed to induce 50% haemolysis. The experimental error is ±5%.

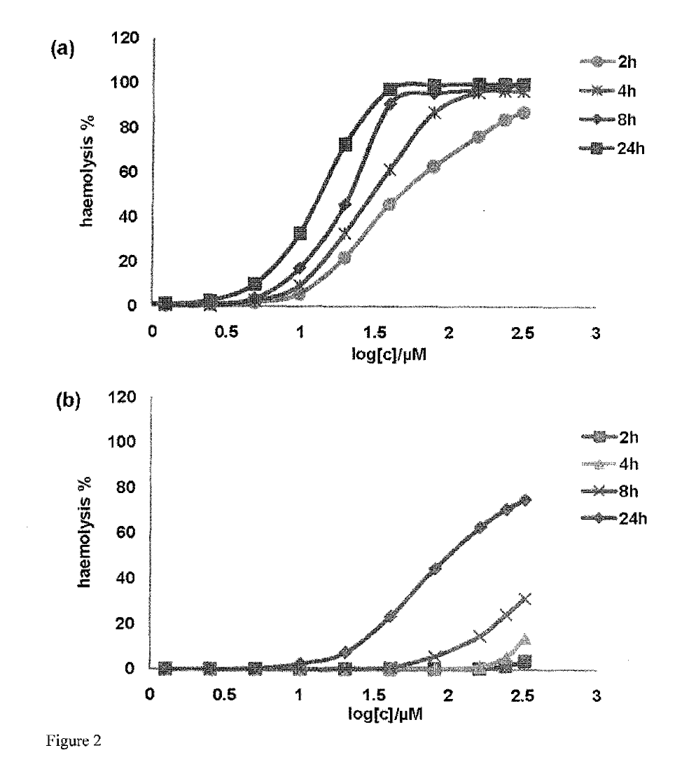

Incubation with ΔΔ-Rubb₁₆ caused the most severe haemolysis and ΔΔ-Rubb₇ the least; however, the HC₅₀ values for all the complexes were significantly higher than the corresponding MIC values. The concentration of the ruthenium complex at the first indication of haemolysis (>2%) was also higher than the MIC values in all cases. Of the complexes tested, ΔΔ-Rubb₁₂ gave the most encouraging results, with excellent antimicrobial activity but low toxicity to human red blood cells, and hence showed the greatest potential for further investigations as an antimicrobial agent. The dose-response curves of ΔΔ-Rubb₁₂ and ΔΔ-Rubb₁₆ for different incubation times are shown in FIG. 2.

The haemolytic activity of ΔΔ-Rubb₁₆ at 10 μM was low for 8 h, but noticeably increased over 24 h. By contrast, ΔΔ-Rubb₁₂ at 10 μM essentially exhibited no haemolysis over a 24 h period. Significant haemolysis was only observed after 4 h at high concentrations (>256 μml⁻¹), while no significant haemolytic activity was observed at all the tested concentrations ΔΔ-Rubb₇ and ΔΔ-Rubb₁₀ over 24 h. In summary, for the haemolytic activity, the Rubbₙ complexes were more concentration-dependent than time-dependent.

### Cytotoxicity Assay

The haemolytic assay indicated that the Rubbₙ dinuclear complexes (especially ΔΔ-Rubb₁₂) were highly active against bacteria but exhibited low toxicity to human red blood cells. However, red blood cells are a special type of eukaryotic cell that do not contain a cell nucleus and the cell membrane has a unique structure of three layers. Given the demonstrated DNA binding ability of several ruthenium complexes (C. Metcalfe and J. A. Thomas, Chem. Soc. Rev., 2003, 32, 215; B. M. Zeglis, V. C. Pierre and J. K. Barton, Chem. Commun., 2007, 4565; and F. R. Keene, J. A. Smith and J. G. Collins, Coord. Chem. Rev., 2009, 253, 2021) it was believed to be important to investigate the toxicity of the complexes against a nucleated eukaryotic cells. Consequently, the toxicity (IC₅₀) against THP-1 cells (a human acute monocytic leukemia cell line and a good model for nucleated eukaryotic cells) was examined (J. Auwerx, Experientia, 1991, 47, 22).

Human monocytic THP-1 cells were obtained from the American Type Culture Collection (ATCC, Rockville, Md., USA). Cells were cultured in RPMI media (Gibco Labs, Grand Island, N.Y., USA) and supplemented with 10% fetal bovine serum (FBS) (Gibco Labs, Grand Island, N.Y., USA), with 20 mM L-glutamine (Sigma Aldrich, Sydney, NSW, Australia), and 12.5 mM HEPES buffer at 37° C. in a humidified atmosphere of 5% CO₂. Cells were seeded at a density of 5×10⁵ per well in a sterile flat bottom 96-well plate, and incubated with the desired drug concentration in duplicate, to a total volume of 200 ml. The concentration range for all the complexes was between 2 mg ml-1 and 1024 mg ml⁻¹. The plates containing each complex were respectively incubated for 24 and 48 h at 37° C. with 5% CO₂. After incubation, the concentration of cells in each well was counted with a haemacytometer and IC₅₀ values were calculated and the results are summarise in Table 6 below:

TABLE 6
The cytotoxicity of ΔΔ-Rubb₇, ΔΔ-Rubb₁₀, ΔΔ-Rubb₁₂ and
ΔΔ-Rubb₁₆ against the THP-1 cell line

| Ruthenium | 24 h | | 48 h | |
|---|---|---|---|---|
| complex | IC₅₀ (μg mL⁻¹) | IC₅₀ [μM] | IC₅₀ (μg mL⁻¹) | IC₅₀ [μM] |
| ΔΔ-Rubb₇ | 400 | 266 | 260 | 173 |
| ΔΔ-Rubb₁₀ | 300 | 194 | 160 | 103 |
| ΔΔ-Rubb₁₂ | 135 | 86 | 90 | 57 |
| ΔΔ-Rubb₁₆ | 78 | 48 | 40 | 25 |

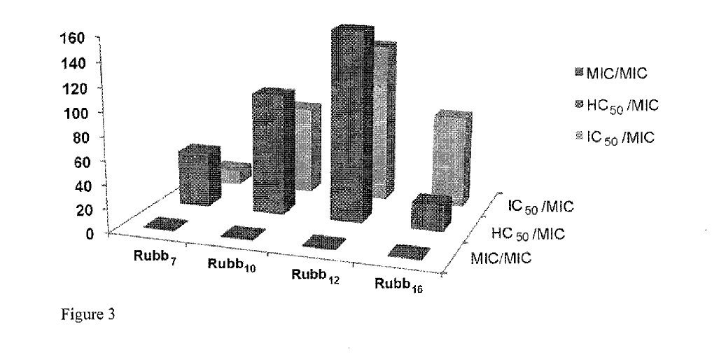

The cytotoxicity of the dinuclear complexes against THP-1 cells increased as the length of the linking chain increased, but again all the complexes exhibited IC₅₀ values greater than their corresponding MICs against susceptible S. aureus and MRSA, as shown in FIG. 3. ΔΔ-Rubb₁₂ displayed the highest HC₅₀/MIC and IC₅₀/MIC ratios, suggesting that it is the most selectively toxic of the Rubbₙ complexes, and consequently has the greatest potential as an antimicrobial agent.

The apparent selectivity of the Rubbₙ complexes for bacterial cells over human cukaryotic cells might be due to a number of reasons. Firstly, bacterial membranes have a higher proportion of negatively-charged phospholipids, whereas, the phospholipids in eukaryotic membranes are predominantly zwitterionic and uncharged. Furthermore, the cell wall of bacteria commonly contain teichoic acids and lipopolysaccharides which are negatively charged. Consequently, due to favourable electrostatic interactions, cationic drugs are preferentially bound to the outer surface of bacterial cells compared to eukaryotic cells.

Alternatively, due to the higher reproduction rate of bacteria compared to human cells, it is possible that the observed selectivity is due to inhibition of the synthesis of important macromolecules, such as DNA, RNA or the cell wall.

### In Vitro Antimalarial Activity

Continuous In Vitro Cultivation of Plasmodium falciparum Strains

The P. falciparum laboratory adapted strains utilised in this project (Table 7) were cultured in vitro and routinely maintained in RPMI-1640-LPLF complete medium, which contained low concentrations of para-amino benzoic acid (0.0005 mg/L) and folic acid (0.01 mg/L).

TABLE 7
Plasmodium falciparum strains used in this project.

| Strain | Origin | Drug Resistance |
|---|---|---|
| D6 | Sierra-Leone, Africa | Sensitive to chloroquine and pyrimethamine |
| W2 | Indochina | Resistant to chloroquine and pyrimethamine |

The low concentration of folic acid in RPMI-1640-LPLF prevents inhibition of the compound if its activity targets the parasite's folate metabolic pathway. Parasites were cultured in human red blood cells (RBCs) in vitro at 37° C. in special gas mixture (5% O₂, 5% CO₂ and 90% N₂) as described in Trager and Jensen (1979) Science 193: 673-675.

### Preparation of Cultivation Medium

Base cultivation medium consisted of 10.4 g/L RPMI-1640-LPLF powder (Gibco BRL), 5.97 g/L HEPES buffer (MP Biomedicals, USA), 2.0 g/L D-glucose (BDH chemicals, Australia), 0.05 g/L hypoxanthine (Sigma, USA) and 40 mg/L gentamycin (Pfizer, Australia). The pH of the medium was adjusted to 6.9 and the solution was filtered using 0.2 μm pore size (AcroCap, Gelman Science, USA). Complete medium was prepared by adding sodium bicarbonate solution (final concentration, 0.21%) and drug-free heat inactivated human plasma obtained from the Australian Red Cross Blood Service (Brisbane) (final concentration, 10%) to the base RPMI-1640-LPLF. RPMI-1640-LPLF complete medium which lacked [3H]-hypoxanthine ([3H]-RPMI-1640-LPLF) was used during the [3H]-hypoxanthine inhibition growth assay to prevent uptake of hypoxanthine by parasites, as radioactive hypoxanthine uptake is measured as a surrogate marker of growth. All complete medium was used within three days of preparation.

### Preparation of Red Blood Cells

Red blood cells (RBC) were required for P. falciparum parasites to proliferate in vitro. O (Rh+) type blood was obtained from the Australian Red Cross Blood Service. The RBC were washed twice in phosphate-buffered saline (PBS) and once in [³H]-RPMI-1640-LPLF complete medium by centrifugation at 1,500×g for 5 minutes. Following the final wash, the haematocrit was measured as the percent of RBC to total culture volume. The haematocrit was adjusted to 50% by removing or adding [3H]-RPMI-LPLF complete medium.

### Continuous Cultivation of Parasites

All P. falciparum strains were grown in RPMI-1640-LPLF complete medium at 4% haematocrit and 1% to 8% parasitaemia at 37° C. in sealed flasks in a gas mixture of 5% O₂, 5% CO₂ and 90% N₂ (BOC Gases, Brisbane, Australia). For drug susceptibility assays, cultures were routinely synchronised when the majority of parasites (>85%) were at ring stage. Synchronisation involved removing the more mature erythrocytic parasite stages by lysis, resulting in the retention only of early trophozoite stages. Synchronisation was performed by resuspending the infected red blood cell (iRBC) pellet in 5 to 10 times its volume of 5% D-sorbitol (Bacto Laboratories Pty. Ltd., Australia) for 5 minutes (Lambros and Vanderberg, 1979 J Parasitol 65: 418-420). The mixture was centrifuged (1,500 rpm for 5 min) and the supernatant removed. The iRBC were washed twice using PBS and once using [³H]-RPMI-LPLF plain medium. Following synchronisation, a new culture was prepared with an initial parasitaemia of 1% in RPMI-LPLF complete medium.

### Evaluation of In Vitro Antimalarial Activity of the Dinuclear Ruthenium(II) Complexes

The in vitro antimalarial activities of two of the dinuclear ruthenium(II) complexes and chloroquine were assessed by exposing P. falciparum strains to ten serially diluted two-fold concentrations of each compound. Parasite growth was measured by uptake of tritiated [³H]-hypoxanthine into newly synthesised parasitic DNA.

### [³H]-Hypoxanthine Growth Inhibition Assay

The [³H]-hypoxanthine growth inhibition assay (Desjardins et al., 1979 Antimicrobial Agents Chemother 16: 710-718) was used to evaluate the in vitro antimalarial activity of the compounds. Briefly, synchronised parasite cultures (>90% rings, 6 to 8 h post invasion) in [³H]-RPMI-LPLF complete medium with 0.5% parasitaemia and 2% haematocrit were exposed to the compounds at ten two-fold concentrations, ranging from 10,000 to 20 nM in 96-well microtitre plates. Chloroquine was used as a reference drug. Uninfected RBCs at 2% haematocrit were used as background controls. For the 96 h exposure period, the plates were incubated in the gas mixture at 37° C. for approximately 48 h, followed by the addition of 0.2 μCi of ³H-hypoxanthine (GE Healthcare, Amersham) in [³H]-RPMI-1640-LPLF to each well and a further 48 h of incubation and then frozen at −20° C. Plates were thawed and harvested using Tomtech Harvester 96 Mach III and radioactive counts were obtained using Wallac TriLux 1450 Microbeta Liquid Scintillation Counter (Perkin Elmer, USA). All assays were performed in triplicate for each strain and at least on two separate occasions.

### In Vitro Inhibition Concentrations of the Dinuclear Ruthenium(II) Complexes

Tritiated hypoxanthine uptake data were analysed in Graphpad Prism V5.0 software (GraphPad Software Inc. USA). The concentrations of the dinuclear ruthenium(II) complexes and chloroquine were transformed into logarithmic values. After subtracting the background values, the data from drug-treated wells were normalised against drug-free control wells. Non-linear regression analysis was carried out of the compound's concentration versus parasitic hypoxanthine incorporation. The in vitro antimalarial activity of the compound is defined as inhibitory concentrations (IC₅₀) and (IC₉₀) that cause 50% and 90% inhibition of parasite growth as determined by measuring [³H]-hypoxanthine incorporation. The averaged results for two replicate experiments are presented in Table 8.

TABLE 8
In vitro antimalarial susceptibility of two dinucelar ruthenium(II) complexes

| | | | D6 | | W2 | |
|---|---|---|---|---|---|---|
| Compound | mg | MWt | IC₅₀ (nM) | IC₉₀ (nM) | IC₅₀ (nM) | IC₉₀ (nM) |
| Rubb₁₂ | 2.54 | 1572 | 733 (28) | 1250 (71) | 739 (118) | 1400 (0.000) |
| [mean (SD)] | | | | | | |
| Rubb₁₆ | 6.31 | 1628 | 226 (13) | 413 (8) | 289 (28) | 444 (29) |
| [mean (SD)] | | | | | | |
| chloroquine | | | 9.8 (0.5) | 12.5 (2.2) | 176.1 (1.5) | 382.9 (152.5) |

The results suggest that the complexes are equally potent against chloroquine sensitive and chloroquine resistant lines.

1. Compound of the following formula: 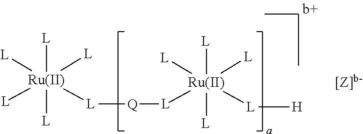
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—;
wherein when the compound does not contain a labile ligand and a=1 then Q contains at least one —NH—, —N(alkyl)- or —O— group.

2. Compound according to claim 1 wherein each L may be the same or different and is independently selected from optionally substituted bipyridine, optionally substituted terpyridine, optionally substituted phenanthroline and labile ligand.

3. Compound according to claim 2 wherein, where present, the or each optional substituent is independently selected from alkyl.

4. Compound according to claim 1 wherein, where present, the or each labile ligand is independently selected from halide or water.

5. Compound according to claim 4 wherein halide is chloride.

6. Compound according to claim 1 wherein Q is selected from C₂₋₁₆alkylene wherein any one or more methylene moieties in alkylene is optionally independently replaced with —NH—, —N(alkyl)- or —O—.

7. Compound according to claim 1 wherein Q is selected from C₂₋₁₆ alkylene.

8. Compound according to claim 1 wherein at least one Ru(II) atom coordinates one labile ligand.

9. Compound according to claim 8 wherein at least one Ru(II) atom does not coordinate one labile ligand.

10. The compound according to claim 1 wherein the two terminal ruthenium centres have the same absolute configuration.

11. The compound according to claim 10 wherein the two terminal ruthenium centres have the Δ-absolute configuration.

12. The compound according to claim 1 of the following formula: 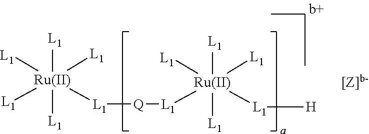
wherein:
a is an integer from 1 to 3, wherein when a is greater than 1 each Q may be the same or different;
b is an integer from 2 to 8;
Z represents one or more counteranions;
each L₁ may be the same or different and is independently selected from pyridyl ligand such that each pyridyl ligand forms a polydentate ligand together with one or more other pyridyl ligands on the same Ru(II) atom;
L₂ is a labile ligand; and
Q is an alkylene linking group wherein any one or more methylene moieties in alkylene is optionally independently replaced with —NH—, —N(alkyl)- or —O—.

13. Method of preventing or treating a microbial infection comprising administering to a subject in need thereof an effective amount of a compound of the following formula: 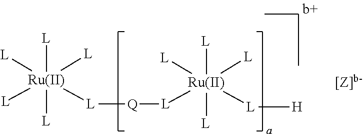
wherein:
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—.

14. Method according to claim 13 wherein at least one Ru(II) atom coordinates one labile ligand.

15. Method according to claim 14 wherein at least one Ru(II) atom does not coordinate one labile ligand.

16. Method according to claim 13 wherein the microbial infection is a bacterial infection.

17. Method according to claim 16 wherein the bacterial infection is a Gram-negative bacterial infection.

18. A compound of the following formula: 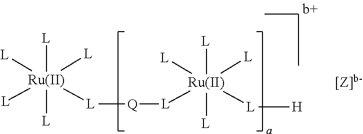
wherein:
a is an integer from 1 to 3, wherein when a is greater than 1 each Q may be the same or different;
b is an integer from 2 to 8;
Z represents one or more counteranions;
each L may be the same or different and is independently selected from pyridyl ligand or labile ligand such that each Ru(II) atom coordinates no more than one labile ligand and each pyridyl ligand forms a polydentate ligand together with one or more other pyridyl ligands on the same Ru(II) atom; and
Q is an alkylene linking group wherein any one or more methylene moieties in alkylene is optionally independently replaced with —NH—, —N(alkyl)- or —O—;
for use in preventing or treating a microbial infection.

19. Compound according to claim 18 wherein at least one Ru(II) atom coordinates one labile ligand.

20.-21. (canceled)

22. Pharmaceutical composition comprising a compound according to claim 1 or a pharmaceutically acceptable salt thereof together with at least one pharmaceutically acceptable carrier or diluent.
